(19) United States
(12) Patent Application Publication
(10) Pub. No.: US 2020/0360367 A1
(43) Pub. Date: Nov. 19, 2020

(54) Treatment of Breast Cancer with Liposomal Irinotecan

(71) Applicant: Ipsen Biopharm Ltd., Wrexham (GB)

(72) Inventors: Eliel Bayever, Cambridge, MA (US); Jonathan Basil Fitzgerald, Arlington, MA (US); Jaeyeon Kim, Lexington, MA (US); Stephan Klinz, Norwood, MA (US)

(73) Assignee: Ipsen Biopharm Ltd., Wrexham (GB)

(21) Appl. No.: 16/711,072

(22) Filed: Dec. 11, 2019

Related U.S. Application Data
(63) Continuation of application No. 14/964,571, filed on Dec. 9, 2015, now abandoned.
(60) Provisional application No. 62/089,685, filed on Dec. 9, 2014.

(51) Int. Cl. A61K 31/4745; A61K 9/127; A61K 9/00

(52) U.S. Cl. CPC A61K 31/4745; A61K 9/1271; A61K 9/0019; A61K 9/1272

(57) ABSTRACT

Provided are methods for treating breast cancer in a patient by administering effective amounts of liposomal irinotecan sucrosofate (MM-398). The breast cancer may be triple negative breast cancer (TNBC), estrogen receptor/progesterone receptor (ER/PR) positive breast cancer, ER-positive breast cancer, or PR-positive breast cancer, or metastatic breast cancer.

### CROSS-REFERENCE TO RELATED APPLICATIONS

This application is a continuation of U.S. patent application Ser. No. 14/964,571, filed Dec. 9, 2015, which claims the benefit of and priority to U.S. Provisional Patent Application No. 62/089,685, filed Dec. 9, 2014, the entire contents of each of which is incorporated herein by reference in their entirety.

### BACKGROUND

Irinotecan (also known as CPT-11) is a highly effective chemotherapeutic agent that, in the form of irinotecan hydrochloride, was approved nearly 20 years ago for the treatment of colorectal cancer. Irinotecan is an active prodrug that is converted in a much more active metabolite known as SN-38 by the action of a carboxylesterase enzyme. In tumors, this carboxylesterase activity is locally concentrated in tumor associated macrophages (TAMs).

MM-398 is a novel liposomally encapsulated preparation of irinotecan sucrosofate. The MM-398 nanoliposomal delivery system is designed to reduce systemic exposure and increase drug accumulation within tumors through the enhanced permeability and retention effect that results from the disorganized and leaky characteristics of tumor vasculature. MM-398 liposomes have been engineered with the aim of optimally exploiting the propensity of TAMs to take up liposomes and to thereby maximize activation of irinotecan to yield intratumoral SN-38. These factors contribute to altering systemic exposure and distribution of MM-398 as compared to irinotecan hydrochloride. Accordingly, safe and effective dosing of MM-398 is not the same as, and its side effect profile differs from that of irinotecan hydrochloride. The altered systemic exposure and distribution of MM-398 is designed to provide an opportunity to administer irinotecan therapy to cancer patients for whom irinotecan hydrochloride cannot be safely dosed in amounts required to provide effective therapy.

One group of cancer patients who would benefit from safe and effective dosing of irinotecan is breast cancer patents, for whom irinotecan hydrochloride has not proven adequately safe and effective to be approved for routine use. The present disclosure provides uses, dosing and administration parameters, methods of use and other factors for treating breast cancer with MM-398, and thereby address the need for new, effective treatments for breast cancer, and provides additional benefits.

### SUMMARY

Provided are methods for treating breast cancer in a patient, the methods comprising administering to the patient liposomal irinotecan (for example, irinotecan sucrose octasulfate salt liposome injection, also referred to as nal-IRI, PEP02, MM-398, or ONIVYDE) according to a particular clinical dosage regimen. Provided too is the use of MM-398 for the safe and effective treatment of breast cancer. Compositions adapted for use in such methods are also provided.

In one aspect, a method for treatment (i.e., effective treatment) of a breast cancer tumor, in a patient (in other words, a use of MM-398) is provided, the method (or use) comprising: administering to the patient an effective amount of liposomal irinotecan in the form of MM-398. In one embodiment, the breast cancer is: a) HER2 negative breast cancer, or b) HER2 negative metastatic breast cancer, or c) HER2 negative or HER2 positive and is metastatic breast cancer with at least one brain lesion. In one embodiment, the brain lesion is a progressive brain lesion. In another embodiment, the administration is carried out in at least one cycle, wherein the cycle is a period of 2 weeks and the irinotecan is administered once per cycle on day 1 of each cycle, and wherein for at least a first cycle the irinotecan is administered at a dose of at least 60 mg/m² or at least 80 mg/m². In one embodiment, the dose is 80 mg/m². In another embodiment, at least the first cycle the irinotecan is administered at a dose of 80, 100, 120, 150, 180, 210, or 240 mg/m². In a particular embodiment, at least the first cycle the irinotecan is administered at a dose of 80 mg/m².

In one embodiment, the administration is carried out in at least two cycles and, if the patient is positive (homozygous) for the UGT1A1*28 allele, the dose following the first cycle is 20 mg/m² or 40 mg/m² lower than the dose given in the first cycle and if the patient is negative for the UGT1A1*28 allele, the dose following the first cycle is the same as the dose given in the first cycle. In another embodiment, all administrations following the first cycle are at the same dose.

In one embodiment, the breast cancer is triple negative or basal-like breast cancer. In another embodiment, the breast cancer is ER-positive, PR-positive, or ER/PR-positive breast cancer. In yet another embodiment, the breast cancer is metastatic breast cancer. In another embodiment, the patient does not have any brain lesions and the breast cancer is HER2 0+ or 1+ by immunohistochemistry, HER2 negative by in situ hybridization, or HER2 negative by dual-probe in situ hybridization. In another embodiment, prior to each administration of the irinotecan, the patient is pre-medicated with either or both of 1) dexamethasone and 2) either a 5-HT3 antagonist or another anti-emetic. In one embodiment, the irinotecan is administered intravenously over 90 minutes. In another embodiment, the administration of the irinotecan, an effective amount of at least one anti-cancer agent other than irinotecan is co-administered to the patient.

In one embodiment, the treatment results in a positive outcome in the patient. In one embodiment, the positive outcome is partial complete response (pCR), complete response (CR), partial response (PR), or stable disease (SD). In another embodiment, the positive outcome is a reduction in: a) tumor size, b) tumor infiltration into peripheral organs, c) tumor metastasis or d) recurrence of tumor. In one embodiment, prior to treatment with the irinotecan, the patient receives a ferumoxytol infusion followed by an MRI scan.

In another aspect is provided a kit for treating a breast cancer in a human patient, the kit comprising a container holding 1) a second container holding at least one dose of MM-398 and 2) instructions for using the irinotecan according to the methods and uses disclosed herein.

### BRIEF DESCRIPTION OF THE FIGURES

FIGS. 1A, 1B, 1C and 1D show images of two ER+ breast cancer patients. FIGS. 1A and 1B are images of a tumor lesion pre-FMX administration and 24 hours post administration (respectively). FIGS. 1C and 1D show a different tumor lesion pre-FMX administration and 24 hours post administration (respectively). The boxed in areas identify the location of the lesion. As can be seen in the figures the lesion in FIGS. 1A and 1B showed low ferumoxytol uptake (lesion did not go dark) This lesion increased in size by 45% following treatment with MM-398. By contrast the lesion in FIGS. 1C and 1D showed high ferumoxytol uptake (lesion went dark) and the lesion size decreased by 49% following treatment with MM-398.

FIG. 2 is a graphical description of the protocol for a Phase 1 study.

FIG. 3A shows FMX levels in individual lesions in 13 patients. Patients 3, 8, and 12 had breast cancer; patient 11 had cervical cancer; patients 2 and 9 had head and neck cancer, patients 7 and 10 had ovarian cancer, patients 4 and 5 had pancreatic cancer, and patients 1, 6, and 13 had other cancers. FIG. 3B shows average FMX kinetics in tumor lesions (●), spleen (▴), muscle (▾), plasma (diamonds), liver (squares).

FIG. 4 shows the correlation between patient's time on the study and the average irinotecan concentration of the biopsied lesion of that patient.

FIGS. 5A, 5B and 5C, are plots showing the correlation between tumor response to MM-398 treatment in lesions showing FMX levels below the median and above the median at 1 hour, 24 hours and 72 hours, respectively plotted against change in tumor size.

FIG. 6A shows a schematic of a FMX tumor PK model was developed using SimBiology® toolbox in MATLAB®. FIG. 6B shows the FMX tumor PK model could quantify the degree of tissue permeability and FMX binding activity across all tumor lesions. FIGS. 6C and 6D show that earlier FMX signals (1 hour and 24 hours) were explained by the model parameters related to vascular permeability.

FIG. 7A provides the time on treatment for various cancer patients and the best overall response as an evaluation after 2 cycles of MM-398 (FIGS. 7B-7F).

FIGS. 8A-8F provide ferumoxytol levels in lesions and PK Model Building: FMX levels in lesions and sub-lesion ROIs are fitted into a PK deposition model that links plasma and lesion values to permeability-surface products (ktrans, kwash-out) and its ratio (Permeability) as well as a binding/retention parameter. Different lesions or sub-lesion areas show distinct PK characteristics. The FMX plasma/lesion ratios show time-dependent parameter correlations. In a preliminary analysis evaluable lesion size changes (CT) from 6 patients are categorized relative to the median of the FMX lesion levels measured at 24 hr.

FIGS. 9A-9D are pictorial representation of the utility of ferumoxytol as a diagnostic test for nal-IRI activity: FMX signals at 1 h and 24 h were used to explore the utility of FMX-MRI as a diagnostic test for nal-IRI in vivo activity in humans. Receiver operating characteristic (ROC) curves were calculated by using two different definitions for responders; 1) Partial Response (PR) in lesion size change (Size Change <−30%) and 2) Decrease in lesion size change (Size Change <0%). Area under curves (AUC) for ROC curves at both time points (1 h and 24 h) were >0.8 suggesting the potential usefulness of FMX-MRI as a diagnostic tool for nal-IRI in vivo activity.

### DETAILED DESCRIPTION

### I. Definitions

As used herein, a “patient” is a human cancer patient.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of a cancer. Effective treatment may refer to alleviation of at least one symptom of a cancer. Such effective treatment may, e.g., reduce patient pain, reduce the size and/or number of lesions, may reduce or prevent metastasis of a cancer tumor, and/or may slow growth of a cancer tumor.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancers, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may do any one or any combination of (i) through (vii) as follows: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop) tumor metastasis; (v) inhibit tumor growth; (vi) prevent or delay occurrence and/or recurrence of tumor; and/or (vii) relieve to some extent one or more of the symptoms associated with the cancer.

The terms “co-administration,” “co-administered,” “concomitant administration” or minor variations of these terms, indicate administration of at least two therapeutic agents to a patient either simultaneously or sequentially within a time period during which the first administered therapeutic agent is still present in the patient when the second administered therapeutic agent is administered.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., taken at a single administration, e.g., orally (e.g., as one, two, three or more pills, capsules, etc.) or injected (e.g., as a bolus). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

“Liposomal Irinotecan” refers to a formulation of the chemotherapy drug irinotecan wherein the irinotecan is encapsulated within a phospholipid bilayer. Examples of liposomal irinotecan include, for example, MM-398 (Merrimack Pharmaceuticals, Inc.) and IHL-305 (Yakult Honsha Co., LTD.).

As used herein, “cancer” refers to a condition characterized by abnormal, unregulated, malignant cell growth. In one embodiment, the cancer is pathologically characterized by a solid tumor, e.g., a breast cancer, e.g., triple negative breast cancer (TNBC, i.e., a breast cancer that is estrogen receptor negative and progesterone receptor negative and HER2 negative), estrogen receptor/progesterone receptor (ER/PR) positive breast cancer, ER-positive breast cancer, or PR-positive breast cancer, or metastatic breast cancer. As used herein, “tumor” and “lesion” are used interchangeably.

The terms “resistant” and “refractory” refer to tumor cells that survive treatment with a therapeutic agent. Such cells may have responded to a therapeutic agent initially, but subsequently exhibited a reduction of responsiveness during treatment, or did not exhibit an adequate response to the therapeutic agent in that the cells continued to proliferate in the course of treatment with the agent. Examples of a resistant or refractory tumor is one where the treatment-free interval following completion of a course of therapy for a patient having the tumor is less than 6 months (e.g., owing to recurrence of the cancer) or where there is tumor progression during the course of therapy.

FERAHEME (ferumoxytol) is a non-stoichiometric magnetite (superparamagnetic iron oxide) coated with polyglucose sorbitol carboxymethylether. The overall colloidal particle size is 17-31 nm in diameter. The chemical formula of ferumoxytol is Fe₅₈₇₄O₈₇₅₂-C₁₁₇₁₉H₁₈₆₈₂O₉₉₃₃Na₄₁₄ with an apparent molecular weight of 750 kDa. An iron replacement product, ferumoxytol is indicated for the treatment of iron deficiency anemia in adult patients with chronic kidney disease.

FERAHEME is an iron replacement product indicated for the treatment of iron deficiency anemia in adult patients with chronic kidney disease (CKD). The recommended dose of FERAHEME for this indication is an initial 510 mg dose followed by a second 510 mg dose 3 to 8 days later. In this context FERAHEME is administered as an undiluted intravenous injection delivered at a rate of up to 1 mL/sec (30 mg/sec). The dosage is expressed in terms of mg of elemental iron, with each mL of FERAHEME containing 30 mg of elemental iron. The hematologic response (hemoglobin, ferritin, iron and transferrin saturation) should be evaluated at least one month following the second FERAHEME injection. The recommended FERAHEME dose may be re-administered to patients with persistent or recurrent iron deficiency anemia. For patients receiving hemodialysis, administer FERAHEME once the blood pressure is stable and the patient has completed at least one hour of hemodialysis. The patient is monitored for signs and symptoms of hypotension following each FERAHEME injection. FERAHEME is contraindicated in patients with evidence of iron overload, known hypersensitivity to FERAHEME or any of its components, and anemia not caused by iron deficiency.

Administration of FERAHEME may transiently affect the diagnostic ability of magnetic resonance (MR) imaging. Anticipated MR imaging studies should be conducted prior to the administration of FERAHEME. Alteration of MR imaging studies may persist for up to 3 months following the last FERAHEME dose. If MR imaging is required within 3 months after FERAHEME administration, T1- or proton density-weighted MR pulse sequences should be used to minimize the FERAHEME effects; MR imaging using T2-weighted pulse sequences should not be performed earlier than 4 weeks after the administration of FERAHEME. Maximum alteration of vascular MR imaging is anticipated to be evident for 1-2 days following FERAHEME administration. FERAHEME will not interfere with X-ray, computed tomography (CT), positron emission tomography (PET), single photon emission computed tomography (SPECT), ultrasound or nuclear medicine imaging.

Although not an approved indication, ferumoxytol is currently being investigated as an imaging agent for the visualization of TAMs and tumor vasculature in cancer patients. Such imaging methods are disclosed, e.g., in co-pending International Publication No. WO2014/113167.

### II. Irinotecan Sucrosofate Liposome Injection (MM-398)

MM-398 is a stable liposomal formulation of irinotecan sucrosofate (irinotecan sucrose octasulfate salt). MM-398 is typically provided as a sterile, injectable parenteral liquid for intravenous injection. The required amount of MM-398 may be diluted, e.g., in 500 mL of 5% dextrose injection USP and infused over a 90 minute period. Additional information on the preparation and use of liposomal irinotecan sucrosofate can be found, e.g., in U.S. Pat. Nos. 8,147,867 and 8,658,203, as well as in WIPO International Application No. PCT/US2013/045495.

An MM-398 liposome is a unilamellar lipid bilayer vesicle of approximately 80-140 nm in diameter that encapsulates an aqueous space which contains irinotecan complexed in a gelated or precipitated state as a salt with sucrose octasulfate. The lipid membrane of the liposome is composed of phosphatidylcholine, cholesterol, and a polyethyleneglycol-derivatized phosphatidyl-ethanolamine in the amount of approximately one polyethyleneglycol (PEG) molecule for 200 phospholipid molecules.

This stable liposomal formulation of irinotecan has several attributes designed to provide an improved therapeutic index. The controlled and sustained release improves activity by increasing duration of exposure of tumor tissue to irinotecan and SN-38. The long circulating pharmacokinetics of MM-398 and its high intravascular drug retention in the liposomes can promote an enhanced permeability and retention (EPR) effect. EPR is believed to promote deposition of liposomes at sites, such as malignant tumors, where the normal integrity of the vasculature (capillaries in particular) is compromised, resulting in leakage out of the capillary lumen of particulates such as liposomes. EPR may thus promote site-specific drug delivery of liposomes to solid tumors. EPR of MM-398 may result in a subsequent depot effect, where liposomes accumulate in tumor associated macrophages (TAMs), which metabolize irinotecan, converting it locally to the substantially more cytotoxic SN-38. This local bioactivation is believed to result in reduced drug exposure at potential sites of toxicity and increased exposure within the tumor.

### III. Irinotecan Glucuronidation

The enzyme produced by the UGT1A1 gene, UDP-glucuronosyltransferase 1, is responsible for bilirubin metabolism and also mediates SN-38 glucuronidation, which is the initial step in the predominant metabolic clearance pathway of this active metabolite of irinotecan. Besides its anti-tumor activity, SN-38 is also responsible for the severe toxicity sometimes associated with irinotecan therapy. Therefore, the glucuronidation of SN-38 to the inactive form, SN-38 glucuronide, is an important step in the modulation of irinotecan toxicity.

Mutational polymorphisms in the promoter of the UGT1A1 gene have been described in which there is a variable number of thymine adenine (ta) repeats. Promoters containing seven thymine adenine (ta) repeats (found in the UGT1A1*28 allele) have been found to be less active than the wild-type promoter (which has six repeats), resulting in reduced expression of UDP-glucuronosyltransferase 1. Patients who carry two deficient alleles of UGT1A1 exhibit reduced glucuronidation of SN-38.

The metabolic transformation of the irinotecan encapsulated in MM-398 to SN-38 includes two critical steps: (1) the release of the irinotecan from the liposome and (2) the conversion of free irinotecan to SN-38. The genetic polymorphisms in humans predictive for the toxicity of irinotecan and those of MM-398 can be considered similar. Nonetheless, due to the smaller tissue distribution, lower clearance and longer elimination half-life of SN-38 of the MM-398 formulation compared to free irinotecan, the deficient genetic polymorphisms may show more association with severe adverse events and/or efficacy.

### IV. Administration

MM-398 is administered by intravenous (IV) infusion over 90 minutes at, e.g., a dose of 80 mg/m² every two weeks in patients not carrying the UGT1A1*28 allele. The first cycle Day 1 is a fixed day; subsequent doses should be administered on the first day of each cycle+/−2 days. As used herein, the dose of MM-398 refers to the dose of irinotecan based on the molecular weight of irinotecan hydrochloride trihydrate unless clearly indicated otherwise.

The dose may also be expressed as the irinotecan free base. Converting a dose based on irinotecan hydrochloride trihydrate to a dose based on irinotecan free base is accomplished by multiplying the dose based on irinotecan hydrochloride trihydrate with the ratio of the molecular weight of irinotecan free base (586.68 g/mol) and the molecular weight of irinotecan hydrochloride trihydrate (677.19 g/mol). This ratio is 0.87 which can be used as a conversion factor. For example, the 80 mg/m² dose based on irinotecan hydrochloride trihydrate is equivalent to a 69.60 mg/m² dose based on irinotecan free base (80×0.87). In the clinic this is rounded to 70 mg/m² to minimize any potential dosing errors. Similarly, a 120 mg/m² dose of irinotecan hydrochloride trihydrate is equivalent to 100 mg/m² of irinotecan free base.

### V. Patient Populations

In one embodiment, a patient treated using the methods and compositions disclosed herein has exhibited evidence of recurrent or persistent breast cancer following primary chemotherapy.

In another embodiment, the patient has had and failed at least one prior platinum based chemotherapy regimen for management of primary or recurrent disease, e.g., a chemotherapy regimen comprising carboplatin, cisplatin, or another organoplatinum compound.

In an additional embodiment, the patient has failed prior treatment with gemcitabine or become resistant to gemcitabine.

The compositions and methods disclosed herein are useful for the treatment of all breast cancers, including breast cancers that are refractory or resistant to other anti-cancer treatments.

### VI. Outcomes

Provided herein are methods for treating breast cancer in a patient, comprising administering to the patient liposomal irinotecan (MM-398) according to a particular clinical dosage regimen.

### Responses to Therapy May Include:

Pathologic complete response (pCR): absence of invasive cancer in the breast and lymph nodes following primary systemic treatment.

Complete Response (CR): Disappearance of all target lesions. Any pathological lymph nodes (whether target or non-target) which has reduction in short axis to <10 mm;

Partial Response (PR): At least a 30% decrease in the sum of dimensions of target lesions, taking as reference the baseline sum diameters;

Stable Disease (SD): Neither sufficient shrinkage to qualify for partial response, nor sufficient increase to qualify for progressive disease, taking as reference the smallest sum diameters while on study; or

Meanwhile, non-CR/Non-PD denotes a persistence of one or more non-target lesion(s) and/or maintenance of tumor marker level above the normal limits.

Progressive Disease (PD) denotes at least a 20% increase in the sum of dimensions of target lesions, taking as reference the smallest sum on study (this includes the baseline sum if that is the smallest on study). In addition to the relative increase of 20%, the sum must also demonstrate an absolute increase of 5 mm. The appearance of one or more new lesions is also considered progression;

In exemplary outcomes, patients treated according to the methods disclosed herein may experience improvement in at least one sign of a breast cancer.

In one embodiment the patient so treated exhibits pCR, CR, PR, or SD.

In another embodiment, the patient so treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, such improvement is measured by a reduction in the quantity and/or size of measurable lesions. Measurable lesions are defined as those that can be accurately measured in at least one dimension (longest diameter is to be recorded) as ≥10 mm by CT scan (CT scan slice thickness no greater than 5 mm), 10 mm caliper measurement by clinical exam or >20 mm by chest X-ray. The size of non-target sites comprising lesions, e.g., pathological lymph nodes can also be measured for improvement. In one embodiment, lesions can be measured on chest x-rays or CT or MRI films.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

In some embodiments, administration of effective amounts of liposomal irinotecan according to any of the methods provided herein produce at least one therapeutic effect selected from the group consisting of reduction in size of a breast tumor, reduction in number of metastatic lesions appearing over time, complete remission, partial remission, stable disease, increase in overall response rate, or a pathologic complete response. In some embodiments, the provided methods of treatment produce a comparable clinical benefit rate (CBR=CR+PR+SD≥6 months) better than that achieved by the same combinations of anti-cancer agents administered without concomitant MM-398 administration.

In other embodiments, the improvement of clinical benefit rate is about 20% 20%, 30%, 40%, 50%, 60%, 70%, 80% or more compared to the same combinations of anti-cancer agents administered without concomitant MM-398 administration.

### Embodiment 1

A method of treatment of a breast cancer in a human patient, the method comprising: administering to the patient an effective amount of liposomal irinotecan, wherein the breast cancer is: a) HER2 negative metastatic breast cancer, or b) HER2 negative or HER2 positive and is metastatic breast cancer with at least one brain lesion.

### Embodiment 2

The method of embodiment 1, wherein the administration is carried out in at least one cycle, wherein the cycle is a period of 2 weeks and the irinotecan is administered once per cycle on day 1 of each cycle, and wherein for at least a first cycle the liposomal irinotecan is administered at a dose of at least 60 mg/m² or at least 80 mg/m².

### Embodiment 3

The method of embodiment 2, wherein for at least the first cycle the liposomal irinotecan is administered at a dose of 80, 100, 120, 150, 180, 210, or 240 mg/m².

### Embodiment 4

The method of embodiment 2 or embodiment 3, wherein for at least the first cycle the liposomal irinotecan is administered at a dose of 80 mg/m².

### Embodiment 5

The method of any one of embodiments 1-4 wherein the administration is carried out in at least two cycles and, if the patient is homozygous for the UGT1A1*28 allele, the dose following the first cycle is 20 mg/m² or 40 mg/m² lower than the dose given in the first cycle and if the patient is not homozygous for the UGT1A1*28 allele, the dose following the first cycle is the same as the dose given in the first cycle.

### Embodiment 6

The method of any one of embodiments 1-5, wherein all administrations following the first cycle are at the same dose.

### Embodiment 7

The method of any one of embodiments 1-6, wherein the breast cancer is triple negative or basal-like breast cancer.

### Embodiment 8

The method of any one of embodiments 1-6, wherein the breast cancer is ER/PR positive breast cancer.

### Embodiment 9

The method of any one of embodiments 1-8, wherein the breast cancer is HER2 negative metastatic breast cancer.

### Embodiment 10

The method of any one of embodiments 1-8, wherein the breast cancer is HER2 negative or HER2 positive metastatic breast cancer with at least one brain lesion and wherein the at least one brain lesion is a progressive lesion.

### Embodiment 11

The method of any one of embodiments 1-9, wherein the patient does not have any brain lesions and the breast cancer is HER2 0+ or 1+ by immunohistochemistry, HER2 negative by in situ hybridization, or HER2 negative by dual-probe in situ hybridization.

### Embodiment 12

The method of any one of embodiments 1-11, wherein, prior to each administration of the liposomal irinotecan, the patient is pre-medicated with either or both of 1) dexamethasone and 2) either a 5-HT3 antagonist or another anti-emetic.

### Embodiment 13

The method of any one of embodiments 1-12, wherein the liposomal irinotecan is administered intravenously over 90 minutes

### Embodiment 14

The method of any one of embodiments 1-13, wherein, concomitant with the administration of the liposomal irinotecan, an effective amount of at least one anti-cancer agent other than irinotecan is co-administered to the patient.

### Embodiment 15

The method of any one of embodiments 1-14, wherein the treatment results in a positive outcome in the patient.

### Embodiment 16

The method of embodiment 15, wherein the positive outcome is pCR, CR, PR, or SD.

### Embodiment 17

The method of embodiment 15, wherein the positive outcome is a reduction in: a) the number of cancer cells, b) tumor size, c) infiltration into peripheral organs, d) tumor metastasis or e) recurrence of tumor.

### Embodiment 18

The method of any one of embodiments 1-17, wherein, prior to treatment with the liposomal irinotecan, the patient receives a ferumoxytol infusion followed by an MRI scan.

### Embodiment 19

The method of any one of embodiments 1-17, wherein the liposomal irinotecan is MM-398.

### Embodiment 20

A kit for treating a breast cancer in a human patient, the kit comprising a container holding 1) a second container holding at least one dose of liposomal irinotecan and 2) instructions for using the liposomal irinotecan according to the method of any one of embodiments 1-18.

### Embodiment 21

The kit according to embodiment 20, wherein the liposomal irinotecan is MM-398.

The following examples are illustrative and should not be construed as limiting the scope of this disclosure in any way; many variations and equivalents will become apparent to those skilled in the art upon reading the present disclosure.

### EXAMPLES

### Example 1: Treatment Protocols

A. Study Design

A clinical trial will enroll patients with metastatic breast cancer in 3 cohorts:
Cohort 1: ER-positive, and PR-positive, or ER/PR-positive breast cancer
Cohort 2: TNBC
Cohort 3: Breast cancer with active brain metastasis
   There are five stages to this study:
1. Screening (−28 d): Patients undergo screening assessments to determine if they are eligible for the study.
2 Ferumoxytol (Day 1-Day 2): patients receive ferumoxytol (FMX) infusion and undergo required MRI (Fe-MRI) scans and pre-treatment biopsy (if applicable, see Cohort requirements) prior to receiving MM-398.
3 MM-398 Treatment (C1D1—progression of disease): Patients receive an MM-398 dose of 80 mg/m² every 2 weeks and other required assessments.
4 Follow up (+30 days from last dose): patients return to clinic 30 days following the last dose of MM-398 for final safety assessments MM-398 will be administered at a dose of 80 mg/m² every two weeks and patients will be treated until disease progression or unacceptable toxicity.
5 Overall survival period: Overall survival (OS) will be collected every month once patients are off study.

B. Patient Selection and Discontinuation

Up to 30 evaluable patients will be enrolled in this study.
I. Inclusion Criteria: In order to be included in the study, patients must have/be:

a) Pathologically confirmed solid tumors that have recurred or progressed following standard therapy, or that have not responded to standard therapy, or for which there is no standard therapy, or who are not candidates for standard therapy.

1. The following invasive breast cancer tumor sub-types are required:
i. Cohorts 1 and 2 must be documented to be HER2 negative as outlined in the ASCO/CAP 2013 guidelines for HER2 testing, defined by at least one of the following:
   HER2 immunohistochemistry (IHC) staining of 0 or 1+, OR if HER2 IHC 2+
   Negative by in situ hybridization (ISH) based on defined as a single-probe average HER2 copy number of less than 4.0 signals/cell.
   OR Negative by Dual-probe ISH defined as a HER2/CEP17 ratio of greater than 2.0 with an average HER2 copy number of fewer than 4.0 signals/cell.
ii. In addition, patients must be able to be categorized into one of the following cohorts:
   Cohort 1: hormone receptor positive breast cancer patients with ER-positive and/or PR-positive tumors defined as >1% of tumor nuclei that are immunoreactive for ER- and/or PR- and HER2-negative
   Cohort 2: triple negative breast cancer (TNBC) patients with ER-negative, PR-negative tumors defined as <1% of tumor nuclei that are immunoreactive for ER and PR and HER2 negative.
   Cohort 3: Any sub-type of metastatic breast cancer and active brain metastases (see additional criteria below).

b) Documented metastatic disease with at least two radiologically measurable lesions as defined by RECIST v1.1 (Eur. J. Cancer 45 (2009) 228-247) (except Cohort 3, see inclusion criteria below)

c) ECOG performance status 0 or 1

d) Bone marrow reserves as evidenced by:
ANC >1,500 cells/μl without the use of hematopoietic growth factors
Platelet count >100,000 cells/μl
Hemoglobin >9 g/dL

e) Adequate hepatic function as evidenced by:
Normal serum total bilirubin
AST and ALT ≤2.5×ULN (≤5×ULN is acceptable if liver metastases are present)

f) Adequate renal function as evidenced by serum creatinine ≤1.5×ULN

g) Normal ECG or ECG without any clinically significant findings

h) Recovered from the effects of any prior surgery, radiotherapy or other anti-neoplastic therapy

i) At least 18 years of age

j) Able to understand and sign an informed consent (or have a legal representative who is able to do so)

### Expansion Phase Additional Inclusion Criteria:

k) Received at least one cytotoxic therapy in the metastatic setting, with exception of TNBC patients who progressed within 12 months of adjuvant therapy

l) Received ≤3 prior lines of chemotherapy in the metastatic setting (no limit to prior lines of hormonal therapy in Cohort 1)

m) Candidate for chemotherapy

n) At least one lesion amenable to multiple pass core biopsy (with the exception of Cohort 3)

The criteria for enrollment must be followed explicitly. Patients will be discontinued from the study treatment in the following circumstances:

### Expansion Phase Cohort 3 Additional Inclusion Criteria:

o) Radiographic evidence of new or progressive brain metastases after prior radiation therapy with at least one brain metastasis measuring ≥1 cm in longest diameter on gadolinium-enhanced MRI (note: progressive brain lesions are not required to meet RECIST v 1.1 criteria in order to be eligible; extra-cranial metastatic disease is also allowed)

p) Imaging following prior radiation is not consistent with pseudo-progression in the judgment of the treating clinician

q) Neurologically stable as defined by:
Stable or decreasing dose of steroids and anti-convulsants for at least 7 days prior to study entry
No clinically significant mass effect, hemorrhage, midline shift, or impending herniation on baseline brain imaging
No significant focal neurologic signs and/or symptoms which would necessitate radiation therapy or surgical decompression, in the judgment of the treating clinician

r) No evidence of diffuse leptomeningeal disease on brain MRI or by previously documented cerebrospinal fluid (CSF) cytology-NOTE: discrete dural metastases are permitted.

II. Exclusion Criteria: Patients must meet all the inclusion criteria listed above and none of the following exclusion criteria:

a) Active central nervous system metastases, indicated by clinical symptoms, cerebral edema, steroid requirement, or progressive disease (applies to Pilot Phase and Expansion Phase Cohorts 1-2 only)

b) Clinically significant gastrointestinal disorder including hepatic disorders, bleeding, inflammation, occlusion, or diarrhea >grade 1
c) Have received irinotecan or bevacizumab (or other anti-VEGF therapy) therapy within the last six months; and for Expansion Phase patients, have received any prior treatment with a Topo1 inhibitor (irinotecan-derived or topotecan)

d) History of any second malignancy in the last 3 years; patients with prior history of in situ cancer or basal or squamous cell skin cancer are eligible. Patients with a history of other malignancies are eligible if they have been continuously disease free for at least 3 years.

e) Unable to undergo MRI due to presence of errant metal, cardiac pacemakers, pain pumps or other MM incompatible devices.

f) A history of allergic reactions to compounds similar to ferumoxytol, as described in full prescribing information for ferumoxytol injection, parenteral iron, dextran, iron-dextran, or parenteral iron-polysaccharide preparations

g) Known hypersensitivity to any of the components of MM-398, or other liposomal products

h) Concurrent illnesses that would be a relative contraindication to trial participation such as active cardiac or liver disease.
Severe arterial thromboembolic events (myocardial infarction, unstable angina pectoris, stroke) less than 6 months before inclusion
NYHA Class III or IV congestive heart failure, ventricular arrhythmias or uncontrolled blood pressure

i) Active infection or an unexplained fever greater than 38.5° C. during screening visits or on the first scheduled day of dosing (at the discretion of the investigator, patients with tumor fever may be enrolled), which in the investigator's opinion might compromise the patient's participation in the trial or affect the study outcome

j) Prior chemotherapy administered within three weeks, or within a time interval less than five half-lives of the agent, whichever is longer, prior to the first scheduled day of dosing in this study

k) Received radiation therapy in the last 14 days

l) Evidence of iron overload as determined by:
Fasting transferrin saturation of >45% and/or
Serum ferritin levels >1000 ng/ml

m) Treated with iron supplements in the previous four weeks

n) HIV-positive patients on combination antiretroviral therapy or other conditions requiring treatment where there is a potential for ferumoxytol to have a negative pharmacokinetic interactions

o) Any other medical or social condition deemed by the Investigator to be likely to interfere with a patient's ability to sign informed consent, to cooperate, and to participate in the study, or to interfere with the interpretation of the results

p) Pregnant or breast feeding; females of child-bearing potential must test negative for pregnancy at the time of enrollment based on a urine or serum pregnancy test. Both male and female patients of reproductive potential must agree to use a reliable method of birth control, during the study and for 3 months following the last dose of study drug.

C. Patient Discontinuation

Patients may withdraw or be withdrawn from the study at any time and for any reason. Some possible reasons for early withdrawal include, but are not limited to the following:
Progressive neoplastic disease
The patient experiences an adverse event which, in the opinion of the Investigator, precludes further participation in the trial.
Clinical and/or symptomatic deterioration
Development of an intercurrent medical condition or need for concomitant treatment that precludes further participation in the trial
Noncompliance with the protocol
Withdraws consent
The Investigator removes the patient from the trial in the best interests of the patient
Study termination by the Sponsor
Use of prohibited concomitant medications
Lost to follow up

If a patient withdraws from the trial, attempts should be made to contact the patient to determine the reason(s) for discontinuation. All procedures and evaluations required by the 30 day follow up visit should be completed when a patient is discontinued. All patients who discontinue the trial as a result of an adverse event must be followed until resolution or stabilization of the adverse event.

D. Description and Use of MM-398

MM-398 is supplied as sterile, single-use vials containing 9.5 mL of MM-398 at a concentration of 5 mg/mL. The vials contain a 0.5 mL excess to facilitate the withdrawal of the label amount from each 10 mL vial.

MM-398 must be stored refrigerated at 2 to 8° C., with protection from light. Light protection is not required during infusion. MM-398 must not be frozen. Responsible individuals should inspect vial contents for particulate matter before and after they withdraw the drug product from a vial into a syringe.

MM-398 must be diluted prior to administration. The diluted solution is physically and chemically stable for 6 hours at room temperature (15-30° C.), but it is preferred to be stored at refrigerated temperatures (2-8° C.), and protected from light. The diluted solution must not be frozen. Because of possible microbial contamination during dilution, it is advisable to use the diluted solution within 24 hours if refrigerated (2-8° C.), and within 6 hours if kept at room temperature (15-30° C.).

Twenty vials of MM-398 will be packaged in a cardboard container. The individual vials, as well as the outside of the cardboard container, will be labeled in accordance with local regulatory requirements.

Dosage and Administration

In one embodiment, MM-398 is dosed and administered as follows.

MM-398 will be administered by intravenous (IV) infusion over 90 minutes at a dose of 80 mg/m² every two weeks. The first cycle Day 1 is a fixed day; subsequent doses should be administered on the first day of each cycle+/−2 days.

Prior to administration, the appropriate dose of MM-398 must be diluted in 5% Dextrose Injection solution (D5W) to a final volume of 500 mL. Care should be taken not to use in-line filters or any diluents other than D5W. MM-398 can be administered at a rate of up to 1 mL/sec (30 mg/sec) using standard PVC-containing intravenous administration bags and tubing.

The actual dose of MM-398 to be administered will be determined by calculating the patient's body surface area at the beginning of each cycle. A +/−5% variance in the calculated total dose will be allowed for ease of dose administration. Since MM-398 vials are single-use vials, site staff must not store any unused portion of a vial for future use and they must discard unused portions of the product.

E. Important Treatment Considerations with MM-398

Data from previous MM-398 studies does not show any unexpected toxicity when compared to the active ingredient, irinotecan, which has been studied extensively. The warnings and precautions for the use of irinotecan and the treatment procedures for managing those toxicities are provided below.

Diarrhea

Irinotecan can induce both early and late forms of diarrhea that appear to be mediated by different mechanisms. Early diarrhea (occurring during or shortly after infusion of irinotecan) is cholinergic in nature. It is usually transient and only infrequently severe. It may be accompanied by symptoms of rhinitis, increased salivation, miosis, lacrimation, diaphoresis, flushing, and intestinal hyper-peristalsis that can cause abdominal cramping. For patients who experienced early cholinergic symptoms during the previous cycle of MM-398, prophylactic administration of atropine will be given at the discretion of the investigator.

Late diarrhea (generally occurring more than 24 hours after administration of irinotecan) can be life threatening since it may be prolonged and may lead to dehydration, electrolyte imbalance, or sepsis. Late diarrhea should be treated promptly with loperamide, and octreotide should be considered if diarrhea persists after loperamide. Loss of fluids and electrolytes associated with persistent or severe diarrhea can result in life threatening dehydration, renal insufficiency, and electrolyte imbalances, and may contribute to cardiovascular morbidity. The risk of infectious complications is increased, which can lead to sepsis in patients with chemotherapy-induced neutropenia. Patients with diarrhea should be carefully monitored, given fluid and electrolyte replacement if they become dehydrated, and given antibiotic support if they develop ileus, fever, or severe neutropenia.

Neutropenia

Deaths due to sepsis following severe neutropenia have been reported in patients treated with irinotecan. Neutropenic complications should be managed promptly with antibiotic support. G-CSF may be used to manage neutropenia, with discretion. Patients, who are known to have experienced Grade 3 or 4 neutropenia while receiving prior anti-neoplastic therapy, should be monitored carefully and managed.

Hypersensitivity

Hypersensitivity reactions including severe anaphylactic or anaphylactoid reactions have been observed. Suspected drugs should be withheld immediately and aggressive therapy should be given if hypersensitivity reactions occur.

Colitis/Ileus

Cases of colitis complicated by ulceration, bleeding, ileus, and infection have been observed. Patients experiencing ileus should receive prompt antibiotic support.

Thromboembolism

Thromboembolic events have been observed in patients receiving irinotecan-containing regimens; the specific cause of these events has not been determined.

Pregnancy

The pregnancy category of irinotecan is D. Women of childbearing potential should be advised to avoid becoming pregnant while receiving treatment with irinotecan. If a pregnancy is reported, treatment should be discontinued. The patient should be withdrawn from the study, and the pregnancy should be followed until the outcome becomes known.

Care of Intravenous Site

Care should be taken to avoid extravasation, and the infusion site should be monitored for signs of inflammation. Should extravasation occur, flushing the site with sterile saline and applications of ice are recommended.

Patients at Particular Risk

In clinical trials of the weekly schedule of irinotecan, it has been noted that patients with modestly elevated baseline serum total bilirubin levels (1.0 to 2.0 mg/dL) have had a significantly greater likelihood of experiencing first-cycle grade 3 or 4 neutropenia than those with bilirubin levels that were less than 1.0 mg/dL (50.0% [ 19/38] versus 17.7% [ 47/226]; p<0.001). Patients with abnormal glucuronidation of bilirubin, such as those with Gilbert's syndrome, may also be at greater risk of myelosuppression when receiving therapy with irinotecan.

Acute Infusion-Associated Reactions

Acute infusion-associated reactions characterized by flushing, shortness of breath, facial swelling, headache, chills, back pain, tightness of chest or throat, and hypotension have been reported in a small number of patients treated with liposome drugs. In most patients, these reactions generally resolve within 24 hours after the infusion is terminated. In some patients, the reaction resolves by slowing the rate of infusion. Most patients who experienced acute infusion reactions to liposome drugs are able to tolerate further infusions without complications.

Other Toxicity Potential

MM-398, the new liposome formulation of irinotecan, is different from irinotecan in unencapsulated formulation, so there is a potential for toxicities other than those caused by irinotecan. All patients should be monitored closely for signs and symptoms indicative of drug toxicity, particularly during the initial administration of treatment.

F. Dose Modification Requirements

Dosing may be held for up to 2 weeks from an occurrence, to allow for recovery from toxicity related to the study treatments. If the time required for recovery from toxicity is more than 2 weeks, the patient should be discontinued from the study, unless the patient is benefiting from the study treatment, in which case the patient's continuation on study should be discussed between Investigator and Sponsor or its designee regarding risks and benefits of continuation.

If a patient's dose is reduced during the study due to toxicity, it should remain reduced for the duration of the study; dose re-escalation to an earlier dose is not permitted. Any patient who has 2 dose reductions and experiences an adverse event that would require a third dose reduction must be discontinued from study treatment.

Infusion reactions will be monitored. Infusion reactions will be defined according to the National Cancer Institute CTCAE (Version 4.0) definition of an allergic reaction/infusion reaction and anaphylaxis, as defined below:

Grade 1: Transient flushing or rash, drug fever <38° C. (<100.4° F.); intervention not indicated
Grade 2: Intervention or infusion interruption indicated; responds promptly to symptomatic treatment (e.g., antihistamines, NSAIDS, narcotics); prophylactic medications indicated for <24 hours.
Grade 3: Symptomatic bronchospasm, with or without urticaria; parenteral intervention indicated; allergy-related edema/angioedema; hypotension
Grade 4: Life-threatening consequences; urgent intervention indicated Study site policies or the following treatment guidelines shall be used for the management of infusion reactions.

### Grade 1

Slow infusion rate by 50%
Monitor patient every 15 minutes for worsening of condition

### Grade 2

### Stop Infusion

Administer diphenhydramine hydrochloride 50 mg IV, acetaminophen 650 mg orally, and oxygen
Resume infusion at 50% of the prior rate once infusion reaction has resolved
Monitor patient every 15 minutes for worsening of condition
For all subsequent infusions, pre-medicate with diphenhydramine hydrochloride 25-50 mg IV

### Grade 3

Stop infusion and disconnect infusion tubing from patient
Administer diphenhydramine hydrochloride 50 mg IV, dexamethasone 10 mg IV, bronchodilators for bronchospasm, and other medications or oxygen as medically necessary
No further treatment with MM-398 will be permitted

### Grade 4

Stop the infusion and disconnect infusion tubing from patient
Administer epinephrine, bronchodilators or oxygen as indicated for bronchospasm
Administer diphenhydramine hydrochloride 50 mg IV, dexamethasone 10 mg IV
Consider hospital admission for observation
No further treatment with MM-398 will be permitted

For patients who experience a Grade 1 or Grade 2 infusion reaction, future infusions may be administered at a reduced rate (over 120 minutes), with discretion.

For patients who experience a second grade 1 or 2 infusion reaction, administer dexamethasone 10 mg IV. All subsequent infusions should be premedicated with diphenhydramine hydrochloride 50 mg IV, dexamethasone 10 mg IV, and acetaminophen 650 mg orally.

G. MM-398 Dose Modifications for Hematological Toxicities

Prior to initiating a new cycle of therapy, the patients must have:
ANC ≥1500/mm³
Platelet count ≥100,000/mm³
   Treatment should be delayed to allow sufficient time for recovery and upon recovery, treatment should be administered according to the guidelines in the tables below. If the patient had febrile neutropenia, the ANC must have resolved to ≥1500/mm³ and the patient must have recovered from infection.

TABLE 1
MM-398 Dose Modifications for Neutrophil Count

| Worst CTCAE | ANC Levels | |
|---|---|---|
| Grade | (cells/mm³) | Modification |
| Grade 1 or 2 | 1000-1999 | Same as previous dose |
| Grade 3 or 4 | <1000 | Reduce dose to 60 mg/m² |
| | | for the first occurrence and to |
| | | 50 mg/m² for the |
| | | second occurrence. |
| | | Patient should be withdrawn if |
| | | reductions lower than |
| | | 50 mg/m² are required. |

TABLE 2
MM-398 Dose Modifications for Other Hematologic Toxicity

| Worst Toxicity | |
|---|---|
| CTCAE Grade | Modification |
| <Grade 2 | Same as previous dose |
| Grade 3 or 4 | Reduce dose to 60 mg/m² |
| | for the first occurrence and to |
| | 50 mg/m² for the |
| | second occurrence. |
| | Patient should be withdrawn if |
| | reductions lower than |
| | 50 mg/m² are required. |

H. MM-398 Dose Modifications for Non-Hematological Toxicities

Treatment should be delayed until diarrhea resolves to ≤Grade 1, and for other Grade 3 or 4 non-hematological toxicities, until they resolve to Grade 1 or baseline. Guidelines for dose adjustment of MM-398 for drug related diarrhea and other Grade 3 or 4 non-hematological toxicities are provided below.

TABLE 3
MM-398 Dose Modifications for Diarrhea

| Worst | | |
|---|---|---|
| Toxicity | | |
| CTCAE | | |
| Grade | Description | Modification |
| Grade 1 | 2-3 stools/day > pretreatment | Same as previous dose |
| Grade 2 | 4-6 stools/day > pretreatment | Same as previous dose |
| Grade 3 | 7-9 stools/day > pretreatment | Reduce dose to 60 mg/m² |
| | | for the first occurrence and to |
| | | 50 mg/m² for the |
| | | second occurrence. |
| | | Patient should be withdrawn if |
| | | reductions lower than |
| | | 50 mg/m² are required. |
| Grade 4 | >10 stools/day > pretreatment | Reduce dose to 60 mg/m² |
| | | for the first occurrence and to |
| | | 50 mg/m² for the |
| | | second occurrence. |
| | | Patient should be withdrawn if |
| | | reductions lower than |
| | | 50 mg/m² are required. |

TABLE 4
MM-398 Dose Modifications for Non-Hematological Toxicities
Other than Diarrhea, Asthenia and Grade 3 Anorexia

| Worst Toxicity | |
|---|---|
| CTCAE Grade | Modification |
| Grade 1 or 2 | Same as previous dose |
| Grade 3 or 4 | Reduce dose to 60 mg/m² |
| (except nausea | for the first occurrence and to |
| and vomiting) | 50 mg/m² for the |
| | second occurrence. |
| | Patient should be withdrawn if |
| | reductions lower than |
| | 50 mg/m² are required. |
| Grade 3 or 4 | Optimize anti-emetic therapy and |
| nausea and/or | reduce dose to 60 mg/m²; |
| vomiting despite | if the patient is already receiving, |
| anti-emetic therapy | for the first occurrence and to |
| | 50 mg/m² for the |
| | second occurrence. |
| | Patient should be withdrawn if |
| | reductions lower than |
| | 50 mg/m² are required. |

I. Concomitant Therapy

All concurrent medical conditions and complications of the underlying malignancy will be treated at the discretion of the Investigator according to acceptable local standards of medical care. Patients should receive analgesics, antiemetics, antibiotics, anti-pyretics, and blood products as necessary. Although warfarin-type anticoagulant therapies are permitted, careful monitoring of coagulation parameters is imperative, in order to avoid complications of any possible drug interactions. All concomitant medications, including transfusions of blood products, will be recorded on the appropriate case report form.

Guidelines for treating certain medical conditions are discussed below; however, institutional guidelines for the treatment of these conditions may also be used. The concomitant therapies that warrant special attention are discussed below.

Antiemetic Medications

Dexamethasone and a 5-HT3 blocker (e.g., ondansetron or granisetron) will be administered to all patients as premedications unless contraindicated for the individual patient. Antiemetics will also be prescribed as clinically indicated during the study period.

Colony Stimulating Factors

Use of granulocyte colony-stimulating factors (G-CSF) is permitted to treat patients with neutropenia or neutropenic fever; prophylactic use of G-CSF will be permitted only in those patients who have had at least one episode of grade 3 or 4 neutropenia or neutropenic fever while receiving study therapy or have had documented grade 3 or 4 neutropenia or neutropenic fever while receiving prior anti-neoplastic therapy.

Therapy for Diarrhea

Acute diarrhea and abdominal cramps, developing during or within 24 hours after MM-398 administration, may occur as part of a cholinergic syndrome. The syndrome will be treated with atropine. Prophylactic or therapeutic administration of atropine should be considered in patients experiencing cholinergic symptoms during the study.

Diarrhea can be debilitating and on rare occasions is potentially life-threatening. Guidelines developed by an ASCO panel for treating chemotherapy-induced diarrhea are abstracted below.

TABLE 5
Management of Chemotherapy Induced Diarrhea

| Clinical | |
|---|---|
| Presentation | Intervention |
| Diarrhea, any grade | Oral loperamide (2 mg every |
| | 2 hours for irinotecan |
| | induced diarrhea): |
| | continue until diarrhea- |
| | free for ≥12 hours |
| Diarrhea persists on | Oral fluoroquinolone × 7 days |
| loperamide for >24 hours | |
| Diarrhea persists on | Stop loperamide; hospitalize |
| loperamide for >48 hours | patient; administer IV fluids |
| ANC <500 cells/μL, | Oral fluoroquinolone (continue |
| regardless of fever | until resolution of neutropenia) |
| or diarrhea | |
| Fever with persistent | Oral fluoroquinolone (continue until |
| diarrhea, even in the | resolution of fever and diarrhea) |
| absence of neutropenia | |

The synthetic octapeptide octreotide has been shown to be effective in the control of diarrhea induced by fluoropyrimidine-based chemotherapy regimens when administered as an escalating dose by continuous infusion or subcutaneous injection. Octreotide can be administered at doses ranging from 100 micrograms twice daily to 500 micrograms three times daily, with a maximum tolerated dose of 2000 micrograms three times daily in a 5-day regimen. Patients should be advised to drink water copiously throughout treatment.

### Other Treatments

Symptomatic treatment for other toxicities should be per institutional guidelines. Prevention of alopecia with cold cap or of stomatitis with iced mouth rinses is allowed.

I. Prohibited Therapy

The following drugs are noted in the irinotecan prescribing information as interacting with irinotecan: St. John's Wort, CYP3A4 inducing anticonvulsants (phenytoin, phenobarbital, and carbamazepine), ketoconazole, itraconazole, troleandomycin, erythromycin, diltiazem and verapamil. Treatment with these agents and any other that interact with irinotecan, should be avoided wherever possible. Because 5-FU interacts with warfarin, caution should be exercised if concomitant use is necessary. Refer to the country specific package inserts of 5-FU and leucovorin for any other drug interactions.

The following therapies are not permitted during the trial:
Other anti-neoplastic therapy, including cytotoxics, targeted agents, endocrine therapy or other antibodies;
Potentially curative radiotherapy; palliative radiotherapy is permitted; and
Any other investigational therapy is not permitted.

J. Laboratory Procedures

Complete Blood Count

A complete blood count (CBC) will be performed locally, and must include a white blood count (WBC) and differential, hemoglobin, hematocrit and platelet count.

Serum Chemistry

Serum chemistry panel will be performed centrally. Additionally, chemistry may also be assessed locally, and local lab results may be used for enrollment and treatment decisions, if central lab results are not available. If local lab results are used for enrollment, then local lab results must be used for all subsequent treatment decisions. Serum chemistry will include electrolytes (sodium, potassium, chloride and bicarbonate), BUN, serum creatinine, glucose, direct and total bilirubin, AST, ALT, alkaline phosphatase, LDH, uric acid, total protein, albumin, calcium, magnesium and phosphate.

Biomarker Samples

Whole blood and plasma will be collected to potentially identify factors that may correlate with tumor response, sensitivity or resistance to MM-398, and MM-398 PK. Non-limiting examples of potential analyses include cytokine levels (e.g., MCSF1 and IL-6), growth factors (e.g., IGF-1 and EGFR family receptors and ligands), and enzyme levels (e.g., MMP9).

Coagulation Profile

A coagulation profile will include a partial thromboplastin time and an international normalized ratio.

UGT1A1*28 Allele

A whole blood sample will be collected from all patients at baseline to test for UGT1A1*28 allele status. The result is not needed prior to the initial dose of MM-398, but subsequent doses of MM-398 may be reduced for patients positive (homozygous) for the UGT1A1*28 allele,

Urine or Serum Pregnancy Test

All women of child bearing potential must undergo a urine or serum pregnancy test.

Pharmacokinetic Assessments

Plasma samples will be collected to determine the levels of MM-398 and SN-38. Additional analytes which may impact the pharmacokinetics of MM-398 may also be measured from this sample. The PK time points outlined in Table 13 below will be drawn during Cycles 1-3.

TABLE 6
Summary of PK Time-points in Treatment and Follow-up Phases

| | Time-point | | |
|---|---|---|---|
| Sample | (Cycles 1-3) | Window | |
| 1 | Immediately prior to | −5 | minutes |
| | MM-398 infusion on Day 1 | | |
| 2 | At the end of the | +5 | minutes |
| | MM-398 infusion | | |
| 3 | +2 hours after the | +/−30 | minutes |
| | completion of the MM-398 infusion | | |
| 4 | +48 hours after the | +/−24 | hours |
| | completion of the MM-398 infusion | | |
| 5 | +168 hours/7 days after the | +/−24 | hours |
| | completion of the MM-398 infusion | | |
| 6 | Immediately prior to | −24 | hours |
| | MM-398 infusion on D 15 | | |

| | | |
|---|---|---|
| 7 | 30 day follow up visit | — |

K. Pain Assessment and Analgesic Consumption

Pain assessment and analgesic consumption diaries will be provided to the patients for recording their pain intensity daily on a visual analogue scale and to document their daily analgesic use.

L. EORTC-QLQ-C30

Quality of life will be assessed by the EORTC-QLQ-C30 instrument. The EORTC-QLQ-C30 is a reliable and valid measure of the quality of life of cancer patients in multicultural clinical research settings. It incorporates nine multi-item scales: five functional scales (physical, role, cognitive, emotional, and social); three symptom scales (fatigue, pain, and nausea and vomiting); and a global health and quality-of-life scale. Several single-item symptom measures are also included.

Patients will be required to complete the EORTC-QLQ-C30 questionnaire at time points outlined in the Schedule of Assessment. On days that the patient is to receive study drug, assessments should be completed prior to study drug administration. Only those patients, for whom validated translations of the EORTC-QLQ-C30 questionnaire are available, will be required to complete the questionnaire.

M. Overall Survival/Post Study Follow-Up

Overall survival data will be collected after a patient completes the 30 day follow-up visit, every 1 month (+/−1 week) from the date of the 30 day follow-up visit. Post-discontinuation data to be collected will include: the date of disease progression (if not already documented; if patient discontinued from study treatment for reasons other than objective disease progression, patient should continue to undergo tumor assessment every 6 weeks, until commencement of new anti-neoplastic therapy or progressive disease); documentation of any anticancer treatment patient has received including the dates of any post-discontinuation systemic therapy, radiotherapy, or surgical intervention; and the date of death. All patients must be followed-up until death or study closure, whichever occurs first.

N. Determining the Severity and Relatedness of Adverse Events

Each adverse event will be graded according to the NCI CTCAE V 4.0, which may be found at http://ctep.cancer.gov/reporting/ctc.html. For events not listed in the CTCAE, severity will be designated as mild, moderate, severe or life threatening or fatal, which correspond to Grades 1, 2, 3, 4 and 5, respectively on the NCI CTCAE, with the following definitions:
Mild: an event not resulting in disability or incapacity and which resolves without intervention;
Moderate: an event not resulting in disability or incapacity but which requires intervention;
Severe: an event resulting in temporary disability or incapacity and which requires intervention;
Life-threatening: an event in which the patient was at risk of death at the time of the event
Fatal: an event that results in the death of the patient

The Investigator must attempt to determine if there exists reasonable possibility that an adverse event is related to the use of the study drug. This relationship should be described as related or non-related.

O. Efficacy Analyses

Progression Free Survival

PFS is defined as the number of months from the date of randomization to the date of death or progression, whichever occurred earlier (per RECIST 1.1). If neither death nor progression is observed during the study, PFS data will be censored at the last valid tumor assessment.

PFS will be compared between the treatment groups using paired un-stratified log-rank tests. The PFS curves will be estimated using Kaplan-Meier estimates. Estimates of the hazard ratios and corresponding 95% confidence intervals will be obtained using Cox proportional hazard models. Stratified analyses will also be carried out using the randomization stratification factors. Treatment effects adjusting for stratification variables and other prognostic covariates will be explored. In addition, different censoring and missing data imputing methods may be used to perform sensitivity analyses on PFS. Methodology for the sensitivity analyses will be fully specified in the Statistical Analysis Plan.

The analyses will be performed for ITT, PP and EP populations.

Time to Treatment Failure

Time to treatment failure is defined as time from randomization to either disease progression, death or study discontinuation due to toxicity. Kaplan-Meier analyses as specified for analyses of progression free survival will be performed for time to treatment failure. The analyses will be performed for ITT, PP and EP populations.

Objective Response Rate

The tumor assessment related to ORR will be determined using RECIST v1.1. If the Sponsor requires an independent review of the radiological assessments to support a new drug application or for any other reason, the response status of all patients may be reviewed by an independent panel of clinicians and may be reviewed by the Sponsor or its designee. In case of a discrepancy between the assessment of the independent panel and that of the investigator, the independent panel's assessment will take precedence.

Objective response rate (ORR) for each treatment group will be calculated combining the number of patients with a best overall response of confirmed CR or PR per RECIST v 1.1. The ORR is the best response recorded from randomization until progression or end of study. The number and percentage of patients experiencing objective response (confirmed CR+PR) at the time of analysis will be presented and the 95% confidence interval for the proportion will be calculated. Objective response rates from the treatment arms will be compared using pair-wise Fisher's Exact Tests. The analyses will be performed for ITT, PP and EP populations.

Tumor Marker Response Analysis

CA 19-9 serum levels will be measured within 7 days before the start of treatment (baseline), and subsequently every 6 weeks. Tumor marker response of CA19-9 will be evaluated by the change of CA19-9 serum levels. Response is defined as a decrease of 50% of CA 19-9 in relation to the baseline level at least once during the treatment period. Only patients with elevated baseline CA 19-9 value (>30 U/mL) will be included in the calculation of tumor marker response rate.

Patient Reported Outcome Analyses

Analysis of the EORTC-QLQ-C30 questionnaires will be performed in accordance with the EORTC guidelines [22].

Safety Analysis

Treatment emergent adverse events will be presented by treatment arm, by patient, by NCI CTCAE grade and by MedDRA system organ class (SOC). Separate listings will be presented for total adverse events, serious adverse events, adverse events related to the study drugs and Grade 3 and 4 adverse events. Laboratory data will be presented by treatment arm and by visit. Abnormal laboratory values will be assessed according to NCI CTCAE grade, where possible. Evaluation of QTc will be done based upon Fridericia's correction method. CTCAE criteria will be applied to the QTc_{F} (i.e. Grade 3=QTc >500 msec). All the safety analyses will be performed by treatment arm, treatment cycle and week, where appropriate. Overall safety will also be evaluated by grade across cycles, SOC and extent of exposure. Additionally, safety analyses will include a comparison between the treatment arms in all patients in the Safety Population:
Number of blood transfusions required
Proportion of patients requiring G-CSF
Adverse events resulting in dose delay or modification

Pharmacokinetics Analysis

Pharmacokinetic data will be collected on all patients randomized to either of the MM-398 arms. Plasma concentration-time data for MM-398 will be analyzed using population pharmacokinetic methods. Pharmacokinetic parameters will be estimated by Non-Linear Mixed Effects Modeling using NONIMIEM®, Version 7, Level 1.0 (ICON Development Solutions, Dublin, Ireland). PK parameters will include plasma Cₘₐₓ, Tₘₐₓ, AUC (area under the concentration curve), clearance, volume of distribution, and terminal elimination half-life. The effects of patient specific factors (age, race, gender, body weight, hepatic and renal function measures, ECOG value, etc.) on pharmacokinetic parameters will be evaluated. Population PK/PD methods will be used to assess the relationships between drug exposure and efficacy and/or toxicity (e.g. neutropenia, diarrhea) parameters.

Additional exploratory analysis may be performed on the PK samples, to help clarify any safety, efficacy or PK issues related to MM-398 that arise during the course of the study. Concentration levels of 5-FU will be summarized descriptively.

### Example 2: Ferumoxytol Magnetic Resonance Imaging

It is anticipated that the MRI parameters will need to be optimized in patients that are enrolled at the beginning of the study and/or in the Expansion Phase, in order to assess any correlations between Fe-MM signal and TAMs, pharmacodynamic markers, or tumor response. Each patient will be required to complete their Fe-MRIs on the same scanner to reduce inter-scan variability. Each MM study will be evaluated for image quality and signal characteristics of tumors and reference tissue on T1-, T2- and T2*-weighted sequences. Once a completed set of images from each patient has been received, the images will be loaded onto the viewing workstation for qualitative review and then sent to a quantitative lab for analysis.

During the Expansion Phase, multiple MR images will be collected on Day 1-Day 2 of the ferumoxytol period, at various time points depending on the scan group to which the patient is assigned. The body areas to be scanned will be determined by the location of the patient's disease; detailed instructions are described in the study imaging manual. All patients will have a baseline image acquired prior to the ferumoxytol infusion, and either a second successive image (baseline repeat; Scan Group 1) or a second image occurring 1-4 h after the end of ferumoxytol administration (Scan Groups 2 and 3). All patients will return on Day 2 for a 24 h Fe-MM using the same protocol and sequences as on Day 1. Patients enrolled into Scan Groups 1 and 2 will require one additional scan either at 24 h or 2 weeks, for a total of 4 scans. Patients will be assigned in an alternating fashion to Scan Groups 1 and 2 before enrollment into Scan Group 3 begins.

TABLE 7
Scan groups and required time points

| | | | Base- | | | | 2 week |
|---|---|---|---|---|---|---|---|
| Scan | | Base- | line | 1-4 | 24 | 24 hours | Base- |
| group | N^{a} | line | (repeat) | hours | hours | (repeat) | line |
| 1 | 5 | X | X | | X | | X |
| 2 | 5 | X | | X | X | X | |
| 3 | 10 | X | | X | X | | |

^{a}Enrollment into Scan Groups 1 and 2 may be increased at the discretion of the Sponsor, in the event that any of the images are not evaluable, or it is determined that more information is needed from the additional scan time points. In this case, enrollment into Scan Group 3 will be decreased by a corresponding number of patients.

TABLE 8
Fe-MRI schedule for Cohort 3 patients
with active brain metastases:

| | | | | | | | |
|---|---|---|---|---|---|---|---|
| | | | Base- | | | | 2 week |
| Scan | | Base- | line | 1-4 | 24 | 24 hours | Base- |
| group | N | line | (repeat) | hours | hours | (repeat) | line |

| | | | | |
|---|---|---|---|---|
| Cohort 3 | 10 | X^{a} | X^{b} | X^{a} |

^{a}Patients with extra-cranial disease will have MRIs of two body areas at baseline and 24 hours: one brain scan and one body scan (body scan will capture the majority of the patient's extra-cranial disease).
^{b}Brain scan only will be completed at this time point

Administration of Ferumoxytol (FERAHEME)

A single dose of ferumoxytol will be administered at Day 1 by intravenous infusion. Dosing is calculated according to patient weight at 5 mg/kg. The total single dose will not exceed 510 mg, the maximum approved single dose of ferumoxytol. Ferumoxytol has in the past been administered as an undiluted IV injection at a rate of up to 1 ml/sec (30 mg/second), with monitoring of vital signs. Alternatively, and in order to mitigate the risk of any toxicity associated with the bolus injection of ferumoxytol, all enrolled patients will receive a single dose of 5 mg/kg of ferumoxytol at Day 1 during the ferumoxytol period by intravenous infusion in 50-200 mL of 0.9% sodium chloride or 5% dextrose over a minimum period of 15 minutes following dilution.

This dosing schedule is less intense than the approved label, which recommends two doses of 510 mg 3 to 8 days apart; however since the use of ferumoxytol as disclosed herein is as an imaging agent, as opposed to a replacement product for iron deficiency, a lower dose is more appropriate.

Ferumoxytol is administered while the patient is in a reclined or semi-reclined position. Patients are closely monitored for signs and symptoms of serious allergic reactions, including monitoring blood pressure and pulse during administration and for at least 30 minutes following each infusion as per the ferumoxytol label instructions.

Important Considerations when Administering Ferumoxytol

Iron levels will be measured in the blood prior to ferumoxytol administration. As currently recommended by the American Association of Liver Disease, screening for iron overload is diagnosed by measuring a fasting morning transferrin saturation ≥45% (ratio of serum iron divided by the serum total iron binding capacity and expressed as a percentage). A ferritin level of 1000 ng/ml is likely to be also associated with organ damaging levels of iron. Both measurement of transferrin saturation and serum ferritin can be altered by inflammation as occurs in malignancy, and may be difficult to interpret. Actual tissue measurement of liver iron is the gold standard for diagnosing iron overload but is associated with some morbidity. Careful interpretation of iron test, preferably by an expert, is recommended.

### Example 3: Physical, Chemical, and Pharmaceutical Properties of MM-398

### Drug Product

The MM-398 drug product contains the drug substance irinotecan in the amount equivalent to 5 mg/mL of irinotecan hydrochloride trihydrate. The drug product liposome is a small unilamellar lipid bilayer vesicle, approximately 110 nm in diameter that encapsulates an aqueous space which contains irinotecan in a gelated or precipitated state, as the sucrosofate salt. The liposome carriers are composed of 1,2-distearoyl-sn-glycero-3-phosphocholine (DSPC), 6.81 mg/mL; cholesterol, 2.22 mg/mL; and methoxy-terminated polyethylene glycol (MW 2000)-distearoylphosphatidylethanolamine (MPEG-2000-DSPE), 0.12 mg/mL. Each mL also contains 2-[4-(2-hydroxyethyl)piperazin-1-yl]lethanesulfonic acid (HEPES) as a buffer, 4.05 mg/mL; sodium chloride as isotonicity reagent, 8.42 mg/mL; and sucrose octasulfate as the drug trapping agent, 0.9 mg/mL. The solution is buffered at pH 7.25. In the vialed product, greater than 98% of the drug is encapsulated in the liposome carrier. MM-398 Injection is supplied as a sterile solution containing 5.0 mg/ml of irinotecan hydrochloride encapsulated in liposomes. The appearance of MM-398 is white to slightly yellow opaque liquid.

### Description and List of Excipients

Table 14 below shows the composition of MM-398 Injection, 5.0 mg/ml drug product. Drug product composition for the 10 mL solution in the vial is also included.

TABLE 14
Quantitative Composition of MM-398 Injection, 5.0 mg/ml

| | Concen- | |
|---|---|---|
| | tration | mg/vial |
| Component | mg/mL | (10 mL) |
| Irinotecan, hydrochloride, trihydrate | 5.0 | 50 |
| Distearoyl phosphatidylcholine (DSPC) | 7.9 | 79 |
| Cholesterol | 2.6 | 26 |
| Pegylated (MW: 2000) Distearoyl | 0.14 | 1.4 |
| phosphatidylethanolamine (PEG 2000 DSPE) | | |
| Sodium chloride | 7.9 | 79 |
| N-2-Hydroxyethylpiperazine-N′-2- | 4.8 | 48 |
| ethanesulfonic acid (HEPES) | | |
| Sodium hydroxide | q.s. to | q.s. to |
| | target | target |
| | pH to 6.5 | pH to 6.5 |
| Water for Injection | q.s. to | q.s. to |
| | 1.0 ml | 10.0 ml |

Abbreviations: MW = molecular weight q.s. = add sufficient quantity.
Note:
DSPC:Cholesterol:PEG 2000 DSPE = 3:2:0.015 (molar ratio)

### Storage Conditions and Shelf Life

Prior to administration, MM-398 Injection must be diluted in 5% Dextrose Injection or Normal Saline (0.9% Sodium Chloride Injection) to a suitable volume for infusion. The solution for infusion (MM-398 Injection and its admixtures) must not be frozen. Freezing will disrupt the liposome structure and result in the release of free irinotecan. Because of the potential for microbial contamination during dilution, the solution for infusion should be used immediately, but may be stored at room temperature (15° to 30° C.) for up to 4 hours prior to the start of the infusion. If necessary, the solution for infusion may be refrigerated (2° to 8° C.) for no more than 24 hours prior to use. MM-398 has been tested for compatibility with limited materials, and no compatibility issues have been identified. The following materials were tested:
Infusion sets (without in-line filter) made of PVC or polyethylene lined
IV bags made of PVC or coextruded film of polyolefin/polyamide
MM-398 drug product must be stored at 2° C. to 8° C.

### Adventitious Agents Safety Evaluation

The only component of biological origin in MM-398 is cholesterol, which is derived from sheep wool. Manufacture of MM-398 uses cholesterol exclusively derived from sheep in New Zealand, where BSE/TSE has not been reported. This material is in compliance with the Note for guidance on minimizing the risk of transmitting animal spongiform encephalopathy agents via human and veterinary medicinal products {EMA/410/01 Rev. 3—March 2011) adopted by the EU Committee for Proprietary Medicinal Products (CPMP) and the Committee for Veterinary Medicinal products (CVMP). The MM-398 cGMP manufacturing process extensively controls for reduction and minimization of bioburden throughout and the drug product is sterile filtered prior to aseptic filling into vials. Product in-process and final testing assures sterility of MM 398.

### Pharmacokinetics and Drug Metabolism in Humans

The pharmacokinetics of MM-398 was evaluated using sample-rich and sparse PK sampling across 6 studies (Study PEP0201, Study PEP0203, Study PEP0206, Study PIST-CRC-01, Study MM-398-01-01-02, and Study MM-398-07-03-01). Both non-compartmental analysis and population pharmacokinetic analysis were performed to evaluate the pharmacokinetic properties of MM-398.

### Pharmacokinetic Parameters

A summary of PK parameters from non-compartmental analysis is provided in Table 22 2 below.

TABLE 22
Summary Statistics of MM-398 NCA Parameters across Multiple PK Studies

| | | Analytes | | | | | |
|---|---|---|---|---|---|---|---|
| | Dose, | Total Irinotecan | | | SN-38 | | |
| PK Parameters | mg/m² | N | Median | % IQR | N | Median | % IQR |
| Cₘₐₓ [μg/ml or | 80 | 25 | 38.0 | 36 | 25 | 4.7 | 89 |
| ng/ml]^{‡} | 120 | 45 | 59.4 | 41 | 45 | 7.2 | 57 |
| t_{1/2} [h] | 80 | 23† | 26.8 | 110 | 13† | 49.3 | 103 |
| | 120 | 45 | 15.6 | 198 | 40† | 57.4 | 67 |
| AUC_{0-∞} [h · μg/ml | 80 | 23† | 1030 | 169 | 13† | 587 | 69 |
| or h · ng/ml]^{‡} | 120 | 45 | 1258 | 192 | 40† | 574 | 64 |
| V_{d} [L/m²] | 80 | 23† | 2.2 | 55 | NA | NA | NA |
| | 120 | 45 | 1.9 | 52 | NA | NA | NA |

†t_{1/2} and AUC_{0-∞} were not calculated for a subset of patients due to insufficient number of samples in the terminal phase.
NA = not available.
Cₘₐₓ are in μg/ml for total irinotecan and ng/ml for SN-38; AUC are in h μg/ml for total irinotecan and h ng/ml for SN-38.

### Population Pharmacokinetics

Population pharmacokinetic analysis was performed for total irinotecan and SN-38 in 353 patients across 6 studies to identify major sources of inter-patient variability and to establish MM-398 exposure-response relationship. The SN-38 originating from the in vivo conversion of released irinotecan was predicted from the model and denoted as “SN-38 Converted”.

From the population pharmacokinetic analysis, total irinotecan was approximately 3 orders of magnitude higher than SN-38. Compared to 120 mg/m² q3w, doses of 80 mg/m² q2w MM-398 resulted in similar average concentration, 1.5-fold lower Cₘₐₓ of both irinotecan and SN-38, and 7-fold higher SN-38 Converted Cₘᵢₙ.

### Example 2

A Phase 1 Study in Patients with Metastatic Breast Cancer to Evaluate Ferumoxytol as a Biomarker for Response to Treatment with MM-398 (Nal-IRI)

MM-398, is designed for extended circulation relative to free irinotecan and to exploit leaky tumor vasculature for enhanced drug delivery to tumors. Preliminary studies show that tumor deposition of nal-IRI and subsequent conversion to SN-38 in both neoplastic cells and tumor associated macrophages (TAM) correlate with response to therapy (lesion size reduction).

A single site pilot study, as further described in Example 3, established the feasibility of performing quantitative FMX MRI. Thirteen patients with advanced cancer (3 with ER/PR+ MBC) were imaged with FMX MRI and treated with nal-IRI. Median tumor lesion FMX uptake in the pilot study was 32.6 and 34.5 ug/mL at 1 h and 24 h, respectively. Lesions with FMX uptake above the median were associated with greater reductions in tumor size following treatment with nal-IRI as determined by CT lesion measurements. The data in this study showing a relationship between FMX levels in tumor lesions and nal-IRI activity provides support for the use of this relationship as a biomarker for nal-IRI deposition and response in solid tumors.

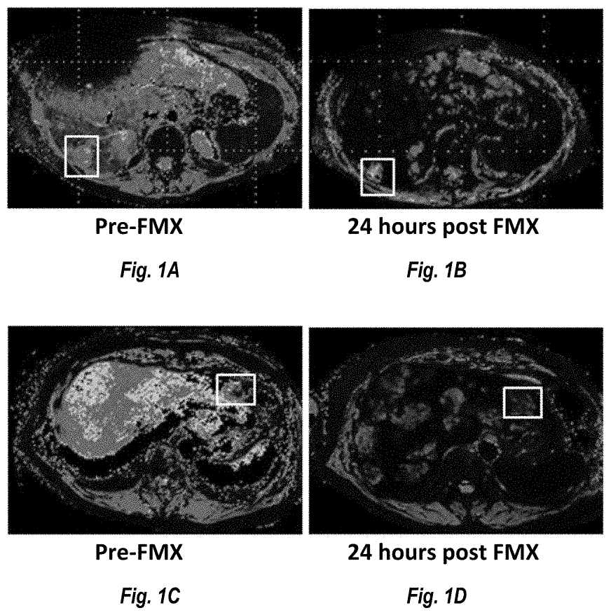

FIGS. 1A-1D shows images of two ER+ breast cancer patients. FIG. 1A and FIG. 1B are images of a tumor lesion pre-FMX administration and 24 hours post administration (respectively). FIG. 1C and FIG. 1D show a different tumor lesion pre-FMX administration and 24 hours post administration (respectively). The boxed in areas identify the location of the lesion. As can be seen in the figures the lesion in FIGS. 1A and 1B showed low ferumoxytol uptake (lesion did not go dark) This lesion increased in size by 45% following treatment with MM-398. By contrast the lesion in FIGS. 1C and 1D showed high ferumoxytol uptake (lesion went dark) and the lesion size decreased by 49% following treatment with MM-398.

Breast Cancer Expansion Study Design

This study has been expanded to include additional MBC patients to further evaluate the technical feasibility of FMX MRI at multiple study sites, and to evaluate activity of nal-IRI in patients with MBC.

Trial Design:

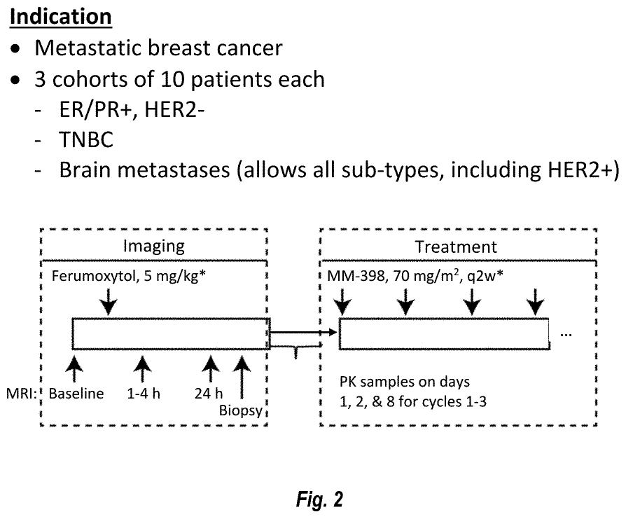

Three cohorts of 10 patients with MBC in the following categories will be enrolled: ER and/or PR positive/HER2-negative, triple negative (TNBC) and MBC with brain metastases. An imaging phase will be followed by a treatment phase. The imaging phase consists of a baseline MM scan, FMX infusion, and follow-up MRI scans at 1-4 and 24 h after infusion. The treatment phase begins 1-6 days after imaging and consists of nal-IRI 80 mg/m² q2w. A pretreatment biopsy is required for correlative studies. The study design is shown graphically in FIG. 2.

Study Objectives:

The primary objective of this multisite expansion is to investigate the feasibility of FMX quantitation in tumor lesions at multiple lesion sites in breast cancer. The secondary objective is to characterize the efficacy of nal-IRI in patients with metastatic breast cancer.

Eligibility Criteria:

Patients with MBC, ECOG 0 or 1 with adequate bone marrow reserve and no prior topoisomerase 1 inhibitor or anti-VEGF treatment. ER and/or PR positive/HER2-negative and TNBC patients must have had 1-3 prior lines of chemotherapy in the metastatic setting and have at least 2 measurable lesions. Patients with brain metastasis must be neurologically stable and have new or progressive brain metastases after prior radiation therapy with at least one lesion measuring ≥1 cm in longest diameter on gadolinium-enhanced MRI.

### Example 3

Lesion Characterization with Ferumoxytol MRI in Patients with Advanced Solid Tumors and Correlation with Treatment Response to MM-398.

Eligible patients (n=15) with previously treated solid tumors with progressive disease had MRI scans prior to and following (11, 24, 72 hours) Ferumoxytol (FMX) infusion. Patients then received nal-IRI (80 mg/m2 q2w) until progression. After MM acquisition, the R2*=1/T2* signal was used to calculate FMX levels in plasma and tumor lesions by comparison to a standard curve. Tumor core biopsies were collected 72 hours after FMX injection and again 72 hours after nal-IRI infusion, yielding two biopsies/lesion for each collection point.

Ferumoxytol (FMX) is an iron-oxide superparamagnetic nanoparticle that has been used off-label for its MM contrast properties. FMX has long-circulating pharmacokinetics and is taken up by TAMs with similar distribution patterns to nal-IRI in preclinical models.

Mill images were acquired on a GE 1.5T MM instrument with a T1 FSPGR series with echo delay times from 1.5-13.2 ms. Slice thickness and spacing was 6 mm×1 mm using a 256×256 matrix. T2* values were extrapolated from each image series by exponential fi of signal intensities. A phantom containing know FMX concentrations from 10-200 μg/ml was included during each MM session and demonstrated a linear relationship between R2*=1/T2* and FMX levels. For each imaging series an R2* map was constructed. FMX levels were calculated for each post-injection time point (post-FMX) after subtraction of baseline values (pre-FMX).

Ferumoxytol Lesion Concentration and Kinetics

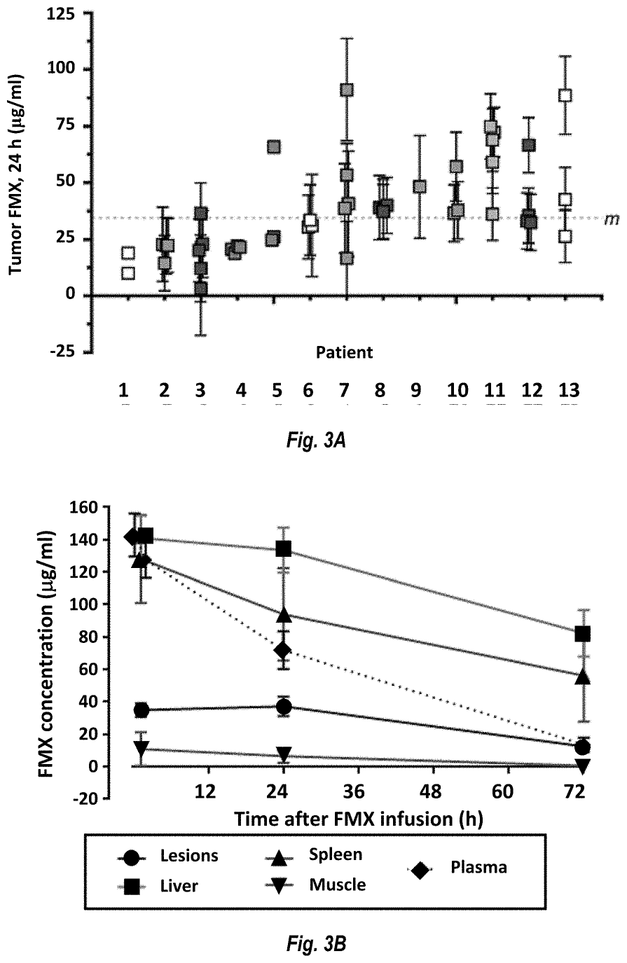

FMX levels were measured in individual lesions from all patients. Lesions within a patient often showed a similar range of uptake levels at 24 hours, and patients could also be ranked according to tumor FMX levels. Error bars are estimated. Median of all lesions (m) is indicated. FIG. 3A shows FMX levels in individual lesions in 13 patients. Patients 3, 8, and 12 had breast cancer; patient 11 had cervical cancer; patients 2 and 9 had head and neck cancer, patients 7 and 10 had ovarian cancer, patients 4 and 5 had pancreatic cancer, and patients 1, 6, and 13 had other cancers. FIG. 3B shows average FMX kinetics in tumor lesions (n=46) and comparison to RES clearance organs (n=11) and normal tissue (n=13) as well as in plasma (n=14).

FMX Signal and Lesion Response Relationships

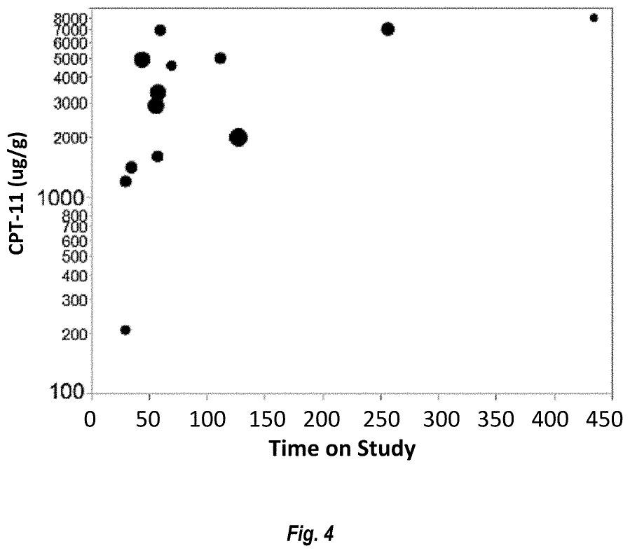

The correlation between patient's time on the study and the average irinotecan concentration of the biopsied lesion of that patient was determined (FIG. 4) (Spearman's r=0.7824; p=0.0016). Biopsies were obtained 72 hours after MM-398 infusion. Time on study is measured from the time of first MM-398 dose.

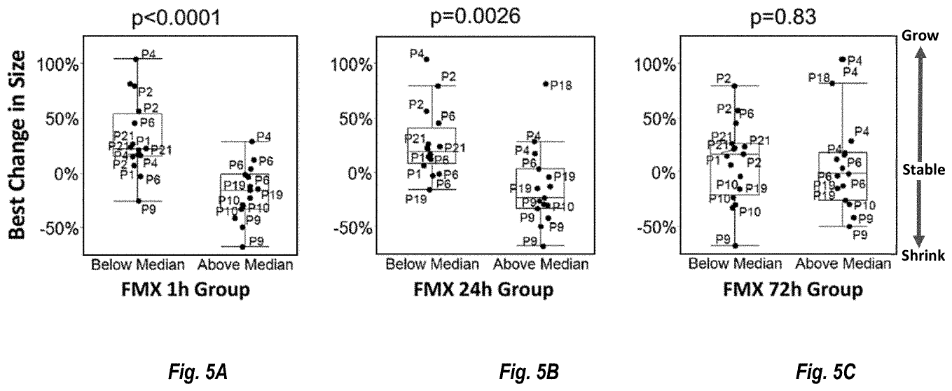

As shown in FIGS. 5A, 5B and 5C, FMX signal correlates with lesion size change. Lesions from each patient were treated as independent samples. FMX signals at each respective time point are grouped relative to the median value observed in the evaluable lesions (9 patients, 31 lesions) and compared to the best change in lesion size seen with RECIST CT. Lesions with FMX levels (in μg/ml) above the population median showed a statistically significant reduction in individual lesion size at early time points (1 hour and 24 hours). No significant lesion response relationship was observed at 72 hours. Lesions from each patient were treated as independent samples.

FMX Deposition and Plasma Clearance

Lesion FMX levels measure 72 hours after FMX injection correlated significantly with MM-398 plasma levels at 72 hours (p=0.7133; p=0.0092) and also with FMX plasma levels at 72 hours (p=0.6154; p=0.332). This may indicate some overlap in the respective clearance processes for FMX and MM-398.

Pharmacokinetic Model of Ferumoxytol

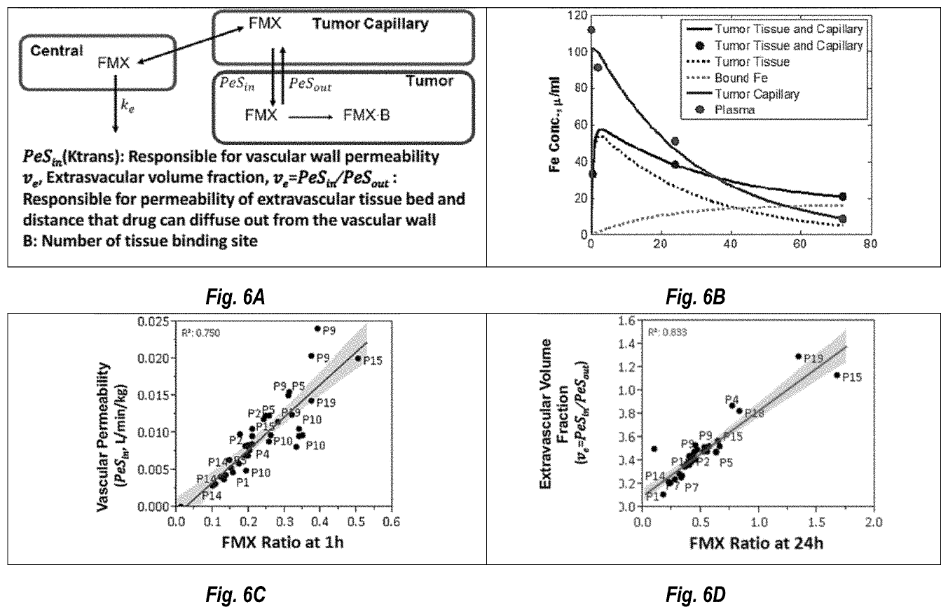

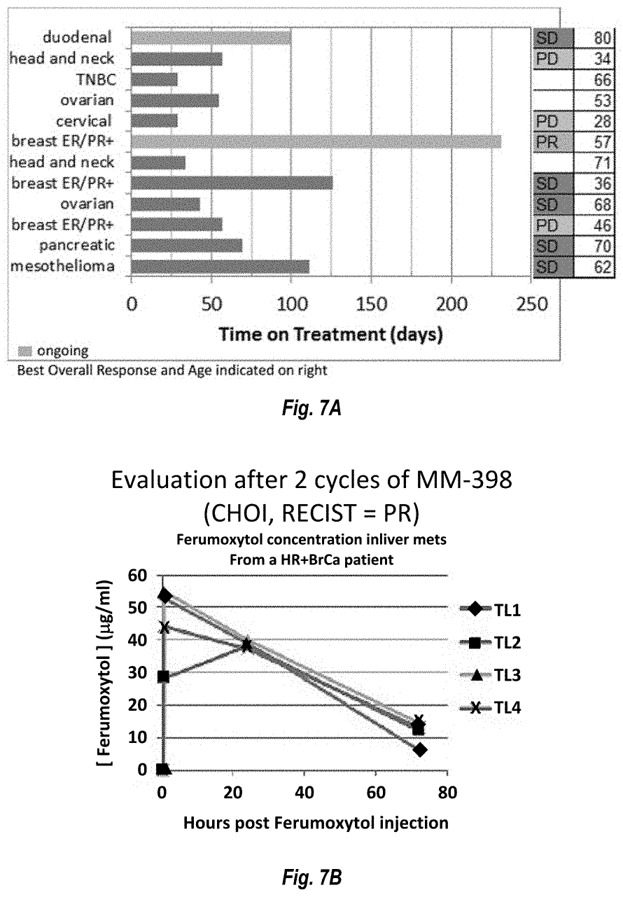

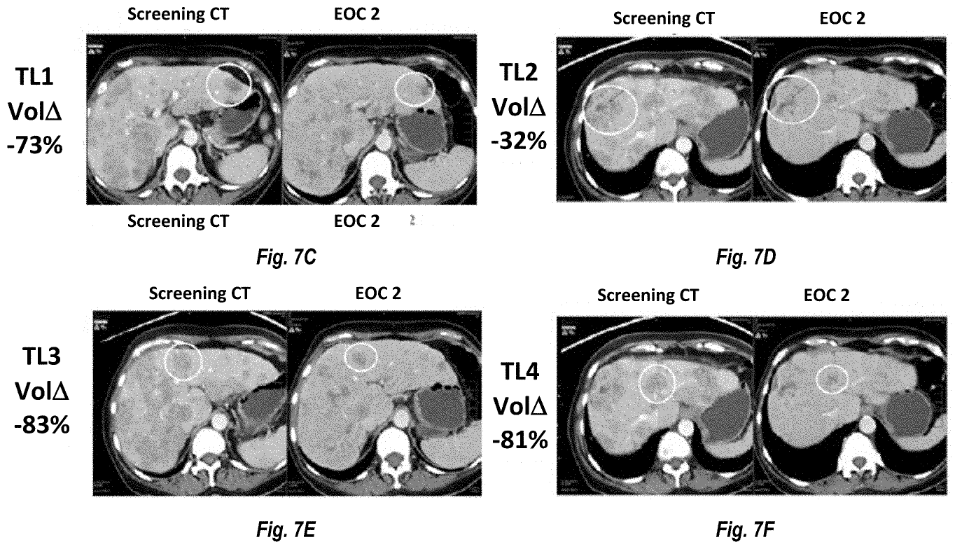

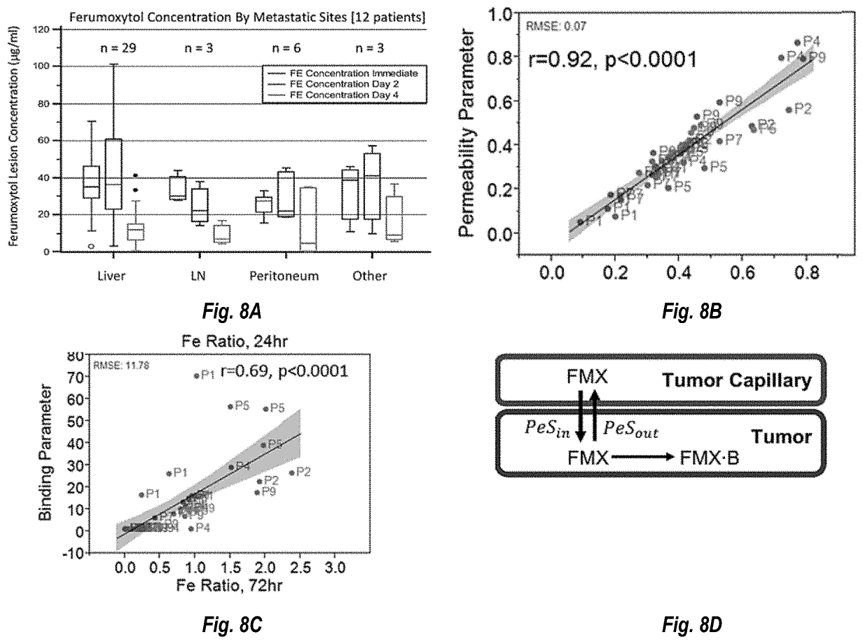

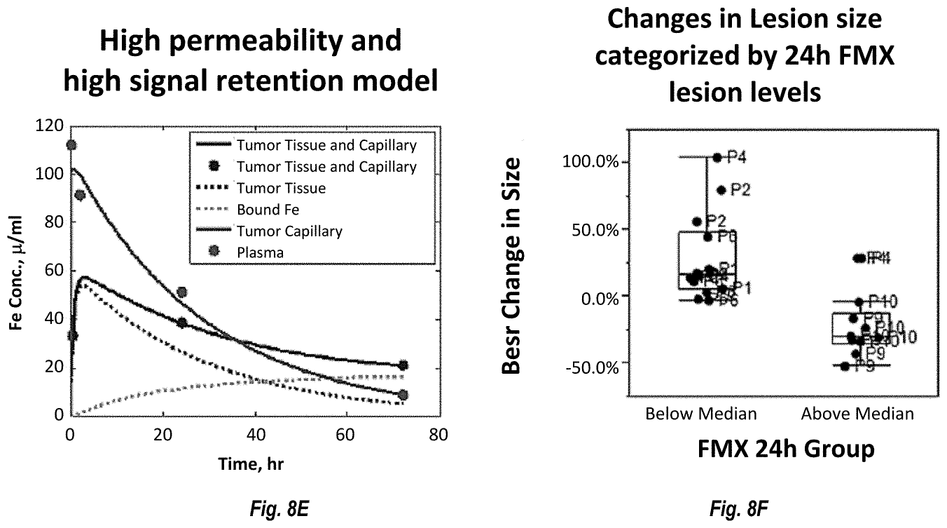

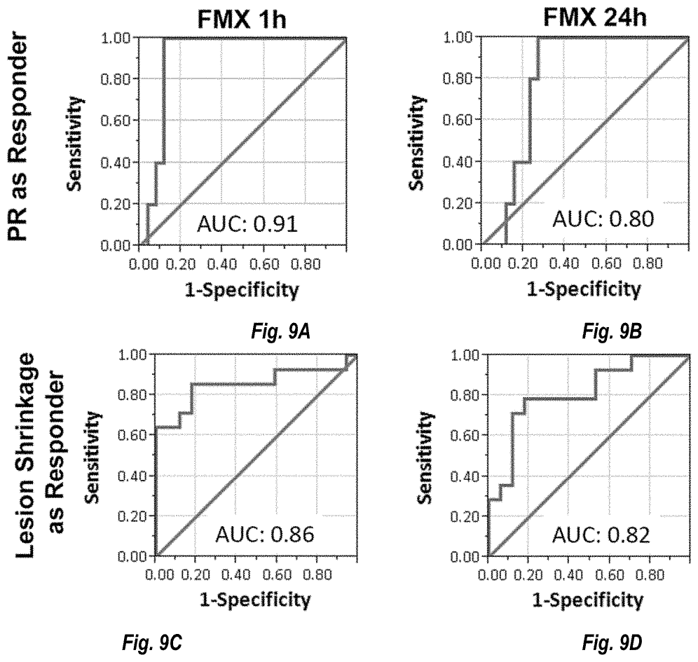

A FMX tumor PK model was developed using SimBiology® toolbox in MATLAB®. A schematic of this model is shown in FIG. 6A. FIG. 6B shows the FMX tumor PK model could quantify the degree of tissue permeability and FMX binding activity across all tumor lesions. FIGS. 6C and 6D show that earlier FMX signals (1 hour and 24 hours) were explained by the model parameters related to vascular permeability. Significantly higher SN-38 levels in a prior study suggested strong local conversion activity of MM-398. Drug and metabolite levels found in the tumor mass concur with the pharmacokinetic modeling expectations.

Summary and Conclusions

Ferumoxytol Mill was able to robustly quantify ferumoxytol levels in plasma as well as normal tissues and tumors. A mechanistic PK model built on these values indicated that tissue permeability to FMX contributed to early FMX MRI signals at 1 hour and 24 hours, while FMX binding contributed at 72 hours. Higher FMX levels, when ranked relative to the median value observed in multiple evaluable lesions from nine patients, were significantly associated with better lesion responses as measured by FMX levels at early time points (p<0.001 at 1 hour post-FMX; p<0.003 at 24 hours).

### Example 4

### Introduction

MM-398, a stable nanoliposomal irinotecan (nal-IRI), is designed to exploit leaky tumor vasculature for enhanced drug delivery to tumors. Tumor deposition of nal-IRI and subsequent irinotecan conversion by CES enzymes in both neoplastic cells and tumor associated macrophages (TAM) may positively correlate with activity. Predictive biomarkers to measure tumor deposition could identify patients likely to benefit from FMX is a 30 nm iron-oxide, superparamagnetic nanoparticle with MM contrast properties. The particle size, its propensity for uptake by TAMs and similar distribution patterns to nal-IRI in preclinical models led to the design of a clinical study to evaluate the feasibility of correlating FMX-based MM (Fe-MM) acquisition with tissue drug metabolite levels and other biomarkers to estimate drug delivery to tumors.

### Patients and Methods

Eligible patients (n=12) with refractory solid tumors with at least two metastatic lesions >2 cm accessible for a percutaneous biopsy were enrolled from one institution. Fe-MM scans were performed on a 1.5T MRI using T2* iron sensitive sequences prior to and following FMX infusion (1 h, 24 h, 72 h). MR images were used to direct biopsies at 72 h to FMX high or low regions, permitting intra- and inter-patient comparisons of FMX and nal-IRI tumor levels. Patients continued on nal-IRI at 80 mg/m² q2w until progression. Tissue iron and TAM distribution were assessed by IHC, tissue-bound metabolite levels by mass-spectrometry. T2* signal was used to calculate FMX levels in total lesions along with FMX estimates on biopsy images derived from fused MRI-CT biopsy images. The first 9 patients (2M 7F; median age 57 years, range 28-71 years) are reported here.

### Results

There were no safety-related or other potential interactions observed with nal-IRI and FMX. Adverse events of nal-IRI were consistent with previous studies. FMX levels, quantified in 36 tumor lesions from the first 9 subjects, showed mean FMX accumulation of 37.9 mcg/mL [3.3-101.2 mcg/mL] and 13.2 mcg/mL [0.1-41.0 mcg/mL] at 24 h and 72 h, respectively. Lesions were localized mostly in liver (67%) and lymph nodes/peritoneal sites (25%). A mechanistic PK model indicated that tissue permeability to FMX contributed to Fe-MM signals at 24 h, while FMX binding contributed at 72 h. Levels of irinotecan and SN-38 were 3.59 mcg/g [2.29-4.89 mcg/g] and 11.43 ng/g [4.04-18.8 ng/g], respectively, at 72 h in biopsies from the first 6 patients.

### Conclusions

This study is one of the first to measure active metabolite SN-38 levels in patient tumors. FMX was safely used as a tumor contrast agent prior to nal-IRI treatment. T2* MRI sequences allowed for quantitation of FMX concentrations in tumor and reference tissue. A mechanistic model provided an estimation of FMX tumor tissue permeability and binding that may be useful as a predictive biomarker of nanotherapeutics such as nal-IRI.

### Study Objectives and Eligibility Criteria

Primary Objectives:
Evaluate the feasibility of Fe-MM to identify TAMs
Measure tumor levels of irinotecan and SN-38

### Secondary Objectives

Correlations between Fe-MM, TAM levels, and tumor levels of irinotecan and SN-38 with administration of nal-IRI
Value of Fe-MM in directing tissue biopsy
Safety profile of nal-IRI in the presence of Ferumoxytol
Assess tumor response to nal-IRI using RECIST 1.1 criteria and volumetric tumor change on CT
Characterize the PK of nal-IRI

### Major Inclusion Criteria:

At least two metastatic lesions >2 cm
Amenable to multiple pass percutaneous biopsies
ECOG performance status 0-2
Bone marrow reserves as evidenced by:
ANC >1,500 cells/μl without the use of hematopoietic growth factors
Platelet count >100,000 cells/μl
Hemoglobin >9 g/dL
Adequate hepatic function as evidenced by:
Normal serum total bilirubin
AST and ALT≤2.5×ULN (≤5×ULN acceptable if liver metastases present)

### Major Exclusion Criteria:

Having received irinotecan or anti-VEGF therapy within the last six months
Unable to undergo MRI imaging due to presence of errant metal, cardiac pacemakers, pain pumps or other MM incompatible devices.
A history of allergic reactions to compounds similar to ferumoxytol
Evidence of Iron overload

### Co-Localization of CD68+ Macrophages and FMX at Stromal Interfaces

Serial tumor sections from FFPE biopsies of liver lesions were assessed by staining with anti-CD68 antibody (clone PG-M1, DAKO) for macrophages and by Prussian Blue staining for FMX. FMX deposition was detectable primarily in stromal areas around tumor nests. The staining pattern suggests intracellular accumulation and is co-localized with macrophages stained in adjacent sections. This association was observed in biopsies obtained at 72 h and 168 h and suggests that FMX deposition can identify vascular-accessible macrophages within tumor lesions.

### Drug Metabolite Quantitation in Tumor Biopsies and Plasma

For tumor tissue analyses, biopsy material averaged 10.5 mg (3.3-21.9 mg). Metabolite detection was in an LC/MS/MS TSQ Vantage instrument. LLoQ was 50 pg/ml for CPT-11 and SN-38G, and 100 pg/ml for SN-38. Plasma analysis of individual metabolites was performed at QPS according to validated procedures. Plasma LLoQ were 140 ng/ml for CPT-11,600 pg/ml for SN-38, and 2.5 ng/ml for SN-38G. These measurements confirmed pharmacokinetic modeling of drug metabolites in plasma and tumor compartments based on prior preclinical and clinical (plasma PK only) observations.

### Cross Indication Translational Study Design

Eligible patients were those with refractory solid tumors in the following indications: NSCLC, CRC, TNBC, ER/PR positive breast cancer, pancreatic cancer, ovarian cancer, gastric cancer, gastro-esophageal junction adenocarcinoma, head and neck cancer. FMX was dosed at 5 mg/kg not to exceed 510 mg total. PK samples for FMX were collected at 0.5 h, 2 h, 24 h and 72 h. nal-IRI was dosed at 80 mg/m2 q2w. PK samples for nal-IRI were collected at 1.5 h, 3.5 h, 72 h and 168 h. Biopsies were targeted towards two separate areas of a lesion, and three passes were collected. Biopsies were obtained 72 h after dosing with either FMX or nal-IRI from separate lesions RECIST v1.1 evaluation every 8 weeks.

### Ferumoxytol Imaging and Quantitation

MRI images were acquired on a GE 1.5T MM instrument with a T1 FSPGR series with echo delay times from 1.5-13.2 ms. Slice thickness and spacing was 6 mm×1 mm using a 256×256 matrix. T2* values were extrapolated from each image series to construct a T2* map. A phantom containing known FMX concentrations from 10-200 mg/ml was included during each MRI session and demonstrated a linear relationship between R2*=1/T2* and FMX levels.
MRI images were taken prior to FMX injection and at 1 h, 24 h and 72 h after injection. FMX levels were calculated for each post injection time point (Post-Fe) after subtraction of baseline values (Pre-Fe). Calculation was done for the complete lesion and for select sub-lesion areas corresponding to biopsy locations.
To measure plasma FMX levels the plasma tubes were placed next to the phantom and imaged in the same instrument. The forgoing procedure provided the means by which tumor Ferumoxytol levels were quantified.

### Conclusions

This phase I study demonstrated the feasibility of incorporating ferumoxytol MRI into a clinical workflow.
No adverse events were attributable to FMX, and phantom evaluation shows that accurate estimates of tumor/tissue Fe concentrations can be obtained with T2* MRI based sequences. FMX tumor PK model successfully described FMX MR signals for each lesion characterizing the information from different time points.
Drug and metabolites are found in the tumor mass and concur with pharmacokinetic modeling expectations.
Prussian Blue staining of ferumoxytol is predominately observed at the stroma-tumor interface and coincides with vascular accessible macrophages.
The correlation between the FMX MM tumor signal and lesion size change was limited by the small sample size of evaluable patients (n=6 at time of data cutoff); if confirmatory, the FMX MRI may be a useful imaging predictive biomarker for liposomal therapies.

### Example 5

### Objectives:

With a systems pharmacology approach we have identified tumor permeability to nal-IRI and ability of tumor carboxylesterase to activate irinotecan as critical factors for in vivo activity. In order to test the importance of these parameters for anti-cancer activity of nal-IRI in patients we have conducted a clinical study to measure and quantify them by using tissue- and imaging-based methods as well as mechanistic PK model.

### Methods:

Eligible patients (n=12) with refractory solid tumors were treated with nal-IRI (80 mg/m2 q2w). Plasma PK was measured at multiple time points, and tissue biopsies were collected 72 h post-treatment, with drug metabolite levels measured by mass spectrometry. Prior to nal-IRI treatment patients underwent ferumoxytol-MRI to test the feasibility to non-invasively measure nanoparticle permeability in tumors. A mechanistic tumor PK model for ferumoxytol was developed to estimate the permeability of ferumoxytol in tumor.

### Results:

Patient-derived data showed that SN-38 concentrations in tumor were 5-fold higher than in plasma 72 h post-treatment in agreement with our simulations incorporating the enhanced permeability and retention effect for tumor deposition of liposomes. The ferumoxytol tumor PK model was able to describe both plasma and tumor ferumoxytol-MRI data (R2>0.9, n=9). Analyses indicated that tumor permeability to ferumoxytol contributed to MM signals at 24 h, while tissue retention capacity of ferumoxytol via binding contributed at 72 h. Ferumoxytol levels above the median were significantly associated with better lesion responses as measured by change in lesion size (p<0.001 at 1 h; p<0.003 at 24 h) resulting in the receiver operating characteristics AUC>0.8 for lesion classification. However, no significant relationship was observed at 72 h.

### Conclusions:

Systems pharmacology approaches can be used to identify parameters of clinical relevance for biomarker development. A promising biomarker strategy for nal-IRI.

### Design of Clinical Translational Study

Eligible patients with refractory solid tumors were recruited. PK samples for FMX were collected at 0.5 h, 2 h, 24 h and 72 h. PK samples for nal-IRI were collected at 1.5 h, 3.5 h, 72 h and 168 h. RECIST v1.1 evaluation was done every 8 weeks.

### Ferumoxytol

Ferumoxytol (FMX) is a 30 nm size superparamagnetic iron oxide nanoparticle coated with polyglucose sorbitol carboxymethylether. FMX is approved for iron supplement in patients with chronic kidney disease and recently has been used as MRI contrast agent (off-label).

### Ferumoxytol Imaging and Quantitation

MR images were acquired on a GE 1.5T MM instrument with a T1 FSPGR series with echo delay times from 1.5-13.2 ms. Slice thickness and spacing was 6 mm×1 mm using a 256×256 matrix. T2* values were extrapolated from each image series to construct a T2* map. Phantom tubes containing known FMX concentrations from 10-200 mg/ml was included during each MRI session and demonstrated a linear relationship between R2*=1/T2* and FMX levels.

### FMX Tumor PK Model Identifies the Temporal Characteristics of FMX Signals

Plasma and tumor PK models were integrated to simulate FMX signals for each patient tumor lesion. FMX tumor PK model was developed by using SimBiology® toolbox in MATLAB®. Particle swarm optimization was used to estimate the model parameters.
Earlier FMX signals (1 h and 24 h) were explained by the model parameters related to vascular permeability, whereas FMX signals at 72 h were explained by the model parameter for FMX binding to tumor tissue.
FMX tumor PK model could quantify the degree of tissue permeability and FMX binding activity across all tumor lesions.

### Plasma and Tumor PK of FMX and Nal-IRI

FMX plasma half-life was similar to nal-IRI as compared to free IRI (A). Even though the estimated tissue permeability parameters for FMX were in between small molecules and liposomes (B), average FMX tumor levels correlated well with nal-IRI deposition to tumor in each patient (C). The mechanistic tumor PK model of nal-IRI predicted higher SN-38 levels in tumor suggesting strong local conversion activity of nal-IRI (D). The predictions were confirmed by the metabolite data from tumor biopsy samples in patients (D, E).

### FMX Signal and Lesion Response Relationship

Lesions with FMX levels above the population median showed statistically significant shrinkage in individual lesion size*. Earlier FMX signals (1 h and 24 h) showed significant lesion response relationship (A,B), whereas no significant relationship was observed at 72 h (C).

### Conclusions

This phase I study demonstrated the feasibility of incorporating FMX-MRI into a clinical workflow.
FMX tumor PK model identified that early FMX signals at 1 h and 24 h contributed to tumor permeability of FMX.
FMX-MRI correlated well with nal-IRI delivery to tumor lesions.
Significantly higher SN-38 levels in tumor suggested strong local conversion activity of nal-IRI Early FMX signals showed significant relationship with lesion size change response suggesting the potential use as a diagnostic tool.

### Endnotes

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

Those skilled in the art will recognize, or be able to ascertain and implement using no more than routine experimentation, many equivalents of the specific embodiments described herein. Such equivalents are intended to be encompassed by the following claims.

Any combinations of the embodiments disclosed in the various dependent claims are contemplated to be within the scope of the disclosure.

The disclosure of each and every U.S., international, or other patent or patent application or publication referred to hereinabove is incorporated herein by reference in its entirety.

1.-6. (canceled)

7. A method of treating a patient having metastatic breast cancer with active brain metastasis comprising intravenously administering an antineoplastic therapy to the patient once every two weeks, the antineoplastic therapy consisting of a 60 mg/m² dose of liposomal irinotecan based on the molecular weight of irinotecan hydrochloride trihydrate.

8. The method of claim 7, wherein the breast cancer is HER2 negative breast cancer.

9. The method of claim 7, wherein the breast cancer is triple negative breast cancer.

10. The method of claim 7, wherein the active brain metastasis is at least one new or progressive brain metastasis after prior radiation therapy.

11. The method of claim 10, wherein the at least one new or progressive brain metastasis is greater than or equal to 1 cm in longest diameter on gadolinium-enhanced magnetic resonance imaging.

12. The method of claim 7, wherein the patient has failed at least one prior platinum-based chemotherapy regimen.

13. The method of claim 7, wherein the patient has failed prior treatment with gemcitabine or has become resistant to gemcitabine.

14. The method of claim 7, wherein prior to each administration of the liposomal irinotecan, the patient is pre-medicated with dexamethasone, an anti-emetic, or both dexamethasone and an anti-emetic.

15. The method of claim 14, wherein the anti-emetic is a 5-HT3 antagonist.

16. The method of claim 7, wherein the liposomal irinotecan is administered intravenously over 90 minutes.

17. The method of claim 7, wherein prior to treatment with the liposomal irinotecan, the patient receives a ferumoxytol infusion followed by a magnetic resonance imaging scan.

18. The method of claim 7, wherein the liposomal irinotecan comprises irinotecan sucrose octasulfate encapsulated in liposomes.

19. The method of claim 7, wherein the liposomal irinotecan comprises irinotecan encapsulated in liposome vesicles in a gelated or precipitated state as a sucrose octasulfate salt.

20. The method of claim 7, wherein the liposomal irinotecan comprises irinotecan encapsulated in liposome vesicles comprising 1,2-distearoyl-sn-glycero-3-phosphocholine (DSPC), cholesterol, and a polyethyleneglycol-derivatized phosphatidyl-ethanolamine.

21. The method of claim 20, wherein the polyethyleneglycol-derivatized phosphatidyl-ethanolamine is methoxy-terminated polyethylene glycol (MW 2000)-distearoylphosphatidylethanolamine (MPEG-2000-DSPE).

22. The method of claim 20, wherein the polyethyleneglycol-derivatized phosphatidyl-ethanolamine is in the amount of approximately one polyethyleneglycol (PEG) molecule for every 200 phospholipid molecules.

23. The method of claim 7, wherein the liposomal irinotecan comprises irinotecan sucrose octasulfate encapsulated in liposomes having a unilamellar lipid bilayer vesicle comprising 6.81 mg/mL 1,2-distearoyl-sn-glycero-3-phosphocholine (DSPC), 2.22 mg/ml cholesterol, and 0.12 mg/ml methoxy-terminated polyethylene glycol (MW 2000)-distearoylphosphatidyl ethanolamine (MPEG-2000-DSPE).
